(19) United States
(12) Patent Application Publication
(10) Pub. No.: US 2022/0348961 A1
(43) Pub. Date: Nov. 3, 2022

(54) CHIMERIC ANTIGEN RECEPTOR CELL LIBRARY CARRYING GENE ELEMENT COMBINATION, PREPRATION AND SCREENING METHOD, AND USE THEREOF

(71) Applicant: PHARCHOICE THERAPEUTICS INC, Shanghai (CN)

(72) Inventors: Shi HU, Shanghai (CN); Wenyan FU, Shanghai (CN)

(73) Assignee: PHARCHOICE THERAPEUTICS INC, Shanghai (CN)

(21) Appl. No.: 17/743,423

(22) Filed: May 12, 2022

Related U.S. Application Data
(63) Continuation of application No. PCT/CN2020/119322, filed on Sep. 30, 2020.

(30) Foreign Application Priority Data
Nov. 13, 2019 (CN) 201911106475.8

(51) Int. Cl. C12N 15/86; C40B 30/06; C40B 40/02; C40B 50/06

(52) U.S. Cl. CPC C12N 15/86; C40B 30/06; C40B 40/02; C40B 50/06; C12N 2740/15043

(57) ABSTRACT

A chimeric antigen receptor (CAR) cell library is established through the fusion of a cell and a vector assembly. The vector assembly carries three genetic elements corresponding to a plurality of first genetic elements encoding one or more idiotype CARs, a second genetic element carrying one or more genetic circuits, and a third genetic element encoding one or more inducible proteins, respectively. The one or more genetic circuits are pre-programmed and are each a combination of a cis-regulatory factor and a transcription factor; and the one or more inducible proteins include one or two selected from the group consisting of a drug resistance protein and a suicide protein. By designing a CAR library-genetic circuit-inducible protein coupling scheme, the cell library construction and screening for complex and unknown disease target antigens are realized, such as to solve the problems that there are complex, diverse, and variable antigens.

### CROSS REFERENCE TO RELATED APPLICATION

This application is a continuation of International Patent Application No. PCT/CN2020/119322 with a filing date of Sep. 30, 2020, designating the United States, now pending, and further claims priority to the Chinese Patent Application No. 201911106475.8 with a filing date of Nov. 13, 2019. The content of the aforementioned applications, including any intervening amendments thereto, are incorporated herein by reference.

### TECHNICAL FIELD

The present disclosure relates to the technical fields of biomedical engineering and synthetic biology, and in particular to a genetic element combination, a chimeric antigen receptor (CAR) cell library carrying the genetic element combination, construction methods of the genetic element combination and the cell library, screening methods for an in vivo antigen and/or an in vitro antigen, and a use of the cell library.

### BACKGROUND ART

The use of genetically-modified immune cells to treat a disease is currently a hot frontier technology. Chimeric antigen receptor-T cells (CAR-T) recognize an antigen expressed on tumor cells through the expression of CAR. CARs are antigen receptors designed to recognize cell surface antigens in a human leukocyte antigen (HLA)-independent manner. The efforts to treat tumor patients with CAR-expressing genetically-modified T cells have made some progress (Molecular Therapy, 2010, 18:4, 666-668; Blood, 2008, 112: 2261-2271). With the continuous development of CAR-T technology, the current CARs can be mainly divided into first-generation CARs, second-generation CARs, and third-generation CARs. The first-generation CAR is composed of an extracellular binding region-single-chain fragment variable (scFV), a transmembrane region (TM), and an intracellular signaling region-immunoreceptor tyrosine-based activation motif (ITAM) that are linked in the form of scFv-TM-CD3. First-generation CAR-T cells can stimulate an anti-tumor cytotoxic effect, but secrete a small amount of cytokines, and thus cannot stimulate a persistent anti-tumor effect in vivo (Zhang T. et al., Cancer Res 2007, 67 (22): 11029-11036.).

The second-generation CAR is developed subsequently by adding an intracellular signaling region of CD28 or CD137 (also known as 4-1BB) on the basis of the first-generation CAR, and the parts of the second-generation CAR are liked in the form of scFv-TM-CD28-ITAM or scFv-TM-CD137-ITAM. The co-stimulation of B7/CD28 or 4-1BBL/CD137 in the intracellular signaling region causes the continuous proliferation of T cells, can increase the levels of cytokines such as IL-2 and IFN-γ secreted by T cells, and can also improve a survival cycle and an anti-tumor effect of CAR-T cells in vivo (Dotti G. et al., CD28 costimulation improves expansion and persistence of chimeric antigen receptor modified T cells in lymphoma patients. J Clin Invest, 2011, 121 (5): 1822-1826).

The third-generation CAR is developed in recent years, and the parts thereof are linked in the form of scFv-TM-CD28-CD137-ITAM or scFv-TM-CD28-CD134-ITAM, which further improves a survival cycle and an anti-tumor effect of CAR-T cells in vivo (Carpenito C. et al., Control of large established tumor xenografts with genetically retargeted human T cells containing CD28 and CD137 domains. PNAS, 2009, 106 (9): 3360-3365.).

In addition, technologies to genetically modify other cells with CAR for therapy are disclosed, such as CAR-NK cells (Choucair K, et al. Future Oncology, 2019, 15 (26): 3053-3069.), CAR NK-92 cells (Schonfeld K, et al. Molecular therapy, 2015, 23 (2): 330-338.), and CAR macrophages (Zhang W, et al., British Journal of Cancer, 2019: 1-9.).

The CAR cell therapy technology has a promising prospect. However, how to determine an antigen-targeted CAR that should be carried by a genetically-engineered cell is an unsolved technical problem for the CAR cell therapy technology. For example, CAR-T cells targeting a single antigen can cause drug resistance, and CAR cells cannot target an unknown antigen.

Major human diseases represented by malignant tumors are characterized by high variability, individual difference, heterogeneity, and evolvability. That is, antigens in lesions of patients with the same disease vary greatly, antigens in different parts of a lesion of a patient are also very different, and lesion cells will evolve after a treatment pressure is applied (for example, radiotherapy, chemotherapy, or the like poses an evolutionary pressure on biological proliferation, such that the gene repair ability of tumor cells is enhanced; and a targeted therapy such as CAR-T therapy or antibody therapy poses an evolutionary pressure on a specific antigen, such that the expression of the related antigen in tumor is down-regulated), which makes it impossible to completely cure a disease. Specific tumor antigens are difficult to identify and have wide diversity and large individual differences; and malignant tumor tissues can undergo biological evolution with the development of conditions and the pressure of treatment, and have high variability. Therefore, if it is intended to treat a tumor with the above method, a process to identify, screen, and prepare a molecule targeting a specific antigen is far behind the progression of the tumor. In addition, due to the huge kurtosis of the human genome, genetic backgrounds of lesions of patients with the same type of malignant tumors are very different. Thus, the therapeutic strategies and therapeutic preparations targeting specific antigens are of no significance for individualized treatment, and evidence-based analysis can only be conducted through marker and genetic research to acquire corresponding populations for which the therapeutic strategies and therapeutic preparations may be effective. The therapy to acquire a tumor antigen through specific tumor tissue analysis and then prepare CAR cells targeting the antigen is also of no significance for individualized treatment.

The patent WO2015/123642 discloses a construction method of a CAR library, where a large number of CARs are prepared to construct a CAR library, and a cell library carrying the CAR library is further constructed. This patent illustrates how to construct a cell library carrying multiple idiotype CARs. However, a limitation of this technology is that a screening direction of the CAR cell library cannot change with antigens, and the screening can only be conducted for specific antigens. The use of a general technique for further screening of the CAR library is described in this patent document, such as the use of the iQue™ screener (Intellicyt, Albuquerque, N. Mex.) (a high-throughput flow cytometer) for high-throughput testing of an individual CAR molecule. However, the process has low efficiency, and the system is designed to quickly acquire available CARs and is not proposed for individualized treatment. For example, through the CAR library in this document, CARs targeting specific antigens can be acquired, but these CARs cannot directly target unknown antigens and also cannot be used as therapeutic products for antigenic diseases with heterogeneity, variability, and evolvability.

In summary, there is an urgent need in the art to establish a CAR cell library with individual properties that overcomes the above-mentioned deficiencies, and a preparation and screening method thereof, which can solve the problem that disease antigens have heterogeneity, variability, and evolvability. In view of this, the present disclosure is specifically proposed.

### SUMMARY

On the basis of the above research background, the present disclosure provides a genetic element combination, a CAR cell library carrying the genetic element combination, construction methods of the genetic element combination and the cell library, screening methods for an in vivo antigen and/or an in vitro antigen, and a use of the cell library.

In a first aspect of the present disclosure, a vector assembly is provided. The vector assembly carries three genetic elements corresponding to: (1) a plurality of first genetic elements encoding one or more idiotype CARs (namely, a CAR library); (2) a second genetic element carrying one or more unique genetic circuits; and (3) a third genetic element encoding one or more unique inducible proteins.

In some preferred embodiments of the present disclosure, there may be at least three idiotype CARs. The one or more idiotype CARs each include an intracellular signaling domain, a transmembrane domain, and an extracellular recognition domain, and the extracellular recognition domain includes an intact antibody, a heavy chain or light chain constituting an antibody, or an antibody fragment; the one or more unique genetic circuits are pre-programmed and are each a combination of a cis-regulatory factor and a transcription factor; and the one or more unique inducible proteins include one or two selected from the group consisting of a drug resistance protein and a suicide protein.

The functions of the genetic elements of the present disclosure can be achieved as follows: when the CARs encoded by the first genetic elements are activated, the pre-programmed genetic circuits of the second genetic element play an expression regulation role for the inducible proteins encoded by the third genetic element. The expression regulation may include any one or a combination of at least two selected from the group consisting of activating transcription and expression, enhancing transcription and expression, terminating transcription and expression, and inhibiting transcription and expression.

In the present disclosure, the term “CAR” used in the first genetic element is an immunotherapeutic concept, and refers to an artificial receptor constructed by imitating an activation process of immune cells. A CAR includes parts of a plurality of immunoreceptors and is designed to recognize an antigen (such as BCR) without any assistance and then directly kill the recognized cell (such as TCR).

A structure of the CAR is a general technique in the art and includes an intracellular signaling domain, a transmembrane domain, and an extracellular recognition domain (an extracellular recognition domain library). The transmembrane domain may further include a hinge region located between the extracellular recognition domain and the transmembrane domain, and one or more additional co-stimulatory molecules located between the transmembrane domain and the intracellular signaling domain.

The intracellular signaling domain may include CD3; the transmembrane domain may include any one selected from the group consisting of a CD28 transmembrane domain, a 4-1BB transmembrane domain, a CD8a transmembrane domain, and a CD3ζ transmembrane domain; the co-stimulatory molecules may include any one or a combination of at least two selected from the group consisting of CD28, CD27, OX40, and 4-1BB; and a structure of the extracellular recognition domain may include, but is not limited to, a library composed of an intact antibody, a chain (heavy or light chain) constituting an antibody, or an antibody fragment (an antibody variable region, a scFV, a single-domain antibody, or an antigen-binding fragment (Fab)), and may preferably include a ScFv library.

The structures of the CARs and the CAR libraries in the present disclosure can be combined in various ways, and resulting combinations can be constructed in the elements of the present disclosure to achieve randomization, as shown in the patent document WO2015/123642.

The extracellular recognition domain library may include a healthy human-derived scFV library, a synthetic scFV library, an alpaca-derived single-domain antibody library, or a sub-library obtained by subtracting an antigen expressed by peripheral blood mononuclear cells (PBMCs) of a subject from a healthy human-derived scFV library. In some specific embodiments of the present disclosure, the extracellular recognition domain library may further include a sub-library obtained by subtracting an antigen expressed by PBMCs and a carcinoembryonic antigen (CEA) of a subject from a healthy human-derived scFV library.

Correspondingly, the CAR library may include, but is not limited to, any one or a combination of at least two selected from the group consisting of an animal-derived library, an immunized animal-derived library, a diseased population-derived library, a healthy population-derived library, a vaccinated population-derived library, a synthetic library, and a library prepared by genetic engineering, and may include a sub-library obtained by pre-processing the above library with an existing technique, such as the sub-library with the background cloning removed described in the non-patent literature [Yin Changcheng, et al., CHINA BIOTECHNOLOGY, 2008, 28 (12): 82-88.].

The extracellular recognition domain library can be further optimized through an antibody engineering process, and the antibody engineering process may include any one or a combination of at least two selected from the group consisting of an antibody affinity maturation technology, an antibody humanization technology, an antibody animalization technology, a multifunctional antibody technology, and a multispecific antibody technology. The affinity maturation technology may include any one or a combination of at least two selected from the group consisting of hotspot site-directed mutagenesis, hotspot random mutagenesis, CDR mutagenesis, strand exchange, and three-dimensional (3D) structure-based antibody mutagenesis.

Preferably, a target targeted by the extracellular recognition domain may include any one or a combination of at least two selected from the group consisting of CD19, BCMA, Mesothelin, GD2, EGFR, HER2, CD22, CD123, Glypican 3, CD30, MUC1, CD33, CD20, CD38, EpCAM, CD56, CD138, CD7, CD133, CEA, CD34, CD117, Claudin18.2, PSCA, cMET, Lewis Y, EphA2, NKG2D ligands, ErbB, NY-ESO-1, CLL-1, CD10, LI13Rα2, CD171, ROR2, AXL, Kappa, CS1, FAP, IL-1RAP, MG7, PSMA, CD5, ROR1, CD70, HER3, Gp75, phosphatidylserine, cMyc, CD4, CD44v6, CD45, CD28, CD3, CD3e, CD52, CD74, CD30, CD166, CD24, EGFR/HER3 fusions, carbohydrates, Aspergillus, Dectin, Ebolavirus, fungi, GP, HERV-K, VEGF-R2, TGF-2R, IgG4, biotin, O-AcGD2, Cadherin 2, OB-cadherin, α5β1 integrin, αVβ6 integrin, Syndecan-1, Cadherin 1, Claudin 12, Claudin 7, Claudin 3, and ZO-1.

Preferably, the extracellular recognition domain library may be constructed from the following monoclonal antibodies (mAbs): ABAGOVOMAB, ABCIXIMAB, ABELACIMAB, ABITUZUMAB, ABREZEKIMAB, ABRILUMAB, ACTOXUMAB, ADALIMUMAB, ADECATUMUMAB, ADUCANUMAB, AFASEVIKUMAB, AFELIMOMAB, ALACIZUMAB, ALEMTUZUMAB, ALIROCUMAB, AMATUXIMAB, ANATUMOMAB, ANDECALIXIMAB, ANETUMAB, ANIFROLUMAB, ANRUKINZUMAB, APRUTUMAB, ASCRINVACUMAB, ASELIZUMAB, ATIDORTOXUMAB, ATINUMAB, ATOLTIVIMAB, ATOROLIMUMAB, AVELUMAB, AZINTUXIZUMAB, BALSTILIMAB, BAPINEUZUMAB, BASILIXIMAB, BAVITUXIMAB, BECTUMOMAB, BEDINVETMAB, BEGELOMAB, BELANTAMAB, BELIMUMAB, BEMARITUZUMAB, BERLIMATOXUMAB, BERSANLIMAB, BERTILIMUMAB, BESILESOMAB, BEVACIZUMAB, BIMAGRUMAB, BIMEKIZUMAB, BIRTAMIMAB, BIVATUZUMAB, BLESELUMAB, BLINATUMOMAB, BLONTUVETMAB, BLOSOZUMAB, BOCOCIZUMAB, BRAZIKUMAB, BRIAKINUMAB, BROLUCIZUMAB, BRONTICTUZUMAB, BUDIGALIMAB, BUROSUMAB, CABIRALIZUMAB, CAMIDANLUMAB, CAMRELIZUMAB, CANAKINUMAB, CANTUZUMAB, CAPLACIZUMAB, CAPROMAB, CARLUMAB, CAROTUXIMAB, CATUMAXOMAB, CEDELIZUMAB, CEMIPLIMAB, CENDAKIMAB, CERGUTUZUMAB, CERTOLIZUMAB, CETRELIMAB, CETUXIMAB, CIBISATAMAB, CINPANEMAB, CITATUZUMAB, CIXUTUMUMAB, CLAZAKIZUMAB, CLENOLIXIMAB, CLIVATUZUMAB, COBOLIMAB, CODRITUZUMAB, COFETUZUMAB, COLTUXIMAB, CONATUMUMAB, CONCIZUMAB, COSFROVIXIMAB, CRENEZUMAB, CRIZANLIZUMAB, CROTEDUMAB, CROVALIMAB, CUSATUZUMAB, DACETUZUMAB, DACLIZUMAB, DALOTUZUMAB, DAPIROLIZUMAB, DECTREKUMAB, DEMCIZUMAB, DENINTUZUMAB, DENOSUMAB, DEPATUXIZUMAB, DETUMOMAB, DEZAMIZUMAB, DILPACIMAB, DINUTUXIMAB, DIRIDAVUMAB, DISITAMAB, DOMAGROZUMAB, DONANEMAB, DORLIMOMAB, DOSTARLIMAB, DROZITUMAB, DULIGOTUZUMAB, DUPILUMAB, DUSIGITUMAB, DUVORTUXIZUMAB, ECROMEXIMAB, EDOBACOMAB, EDRECOLOMAB, EFALIZUMAB, EFUNGUMAB, ELDELUMAB, ELEZANUMAB, ELGEMTUMAB, ELIPOVIMAB, ELSILIMOMAB, EMACTUZUMAB, EMIBETUZUMAB, EMICIZUMAB, ENAPOTAMAB, ENAVATUZUMAB, ENFORTUMAB, ENLIMOMAB, ENOBLITUZUMAB, ENOKIZUMAB, ENOTICUMAB, ENSITUXIMAB, ENVAFOLIMAB, EPITUMOMAB, EPTINEZUMAB, ERLIZUMAB, ERTUMAXOMAB, ETIGILIMAB, ETOKIMAB, ETROLIZUMAB, EVINACUMAB, EXBIVIRUMAB, FARALIMOMAB, FARICIMAB, FARLETUZUMAB, FASINUMAB, FELVIZUMAB, FEZAKINUMAB, FICLATUZUMAB, FIGITUMUMAB, FIRIVUMAB, FLANVOTUMAB, FLETIKUMAB, FLOTETUZUMAB, FONTOLIZUMAB, FORALUMAB, FORAVIRUMAB, FRESOLIMUMAB, FROVOCIMAB, FRUNEVETMAB, FULRANUMAB, FUTUXIMAB, GALIXIMAB, GANCOTAMAB, GANITUMAB, GANTENERUMAB, GARADACIMAB, GARETOSMAB, GAVILIMOMAB, GEDIVUMAB, GEMTUZUMAB, GEVOKIZUMAB, GILVETMAB, GIMSILUMAB, GIRENTUXIMAB, GLEMBATUMUMAB, GLENZOCIMAB, GOLIMUMAB, GOSURANEMAB, IANALUMAB, IBRITUMOMAB, ICRUCUMAB, IDARUCIZUMAB, IERAMILIMAB, IFABOTUZUMAB, IGOVOMAB, ILADATUZUMAB, IMALUMAB, IMAPRELIMAB, IMCIROMAB, IMGATUZUMAB, INCLACUMAB, INDATUXIMAB, INDUSATUMAB, INEBILIZUMAB, INFLIXIMAB, INOLIMOMAB, INOTUZUMAB, INTETUMUMAB, APAMISTAMAB, DERLOTUXIMAB, IPILIMUMAB, IRATUMUMAB, ISATUXIMAB, ISCALIMAB, ISTIRATUMAB, IXEKIZUMAB, KELIXIMAB, LABETUZUMAB, LACNOTUZUMAB, LACUTAMAB, LADIRATUZUMAB, LAMPALIZUMAB, LANADELUMAB, LANDOGROZUMAB, LAPRITUXIMAB, LARCAVIXIMAB, LEBRIKIZUMAB, LEMALESOMAB, LENVERVIMAB, LENZILUMAB, LERDELIMUMAB, LERONLIMAB, LESOFAVUMAB, LETOLIZUMAB, LEVILIMAB, LEXATUMUMAB, LIBIVIRUMAB, LIFASTUZUMAB, LIGELIZUMAB, LILOTOMAB, LINTUZUMAB, LIRILUMAB, LODELCIZUMAB, LONCASTUXIMAB, LORVOTUZUMAB, LOSATUXIZUMAB, LUCATUMUMAB, LULIZUMAB, LUMILIXIMAB, LUMRETUZUMAB, LUPARTUMAB, LUTIKIZUMAB, MAFTIVIMAB, MAGROLIMAB, MAPATUMUMAB, MARGETUXIMAB, MARSTACIMAB, MASLIMOMAB, MATUZUMAB, MAVRILIMUMAB, MEPOLIZUMAB, METELIMUMAB, MILATUZUMAB, MINRETUMOMAB, MIRIKIZUMAB, MIRVETUXIMAB, MITAZALIMAB, MITUMOMAB, MODOTUXIMAB, MOGAMULIZUMAB, MONALIZUMAB, MOROLIMUMAB, MOSUNETUZUMAB, MOTAVIZUMAB, MURLENTAMAB, NACOLOMAB, NAMILUMAB, NAPTUMOMAB, NARATUXIMAB, NARNATUMAB, NATALIZUMAB, NAVICIXIZUMAB, NAVIVUMAB, NAXITAMAB, NEBACUMAB, NEMOLIZUMAB, NERELIMOMAB, NESVACUMAB, NETAKIMAB, NIDANILIMAB, NIMACIMAB, NIMOTUZUMAB, NIRSEVIMAB, NIVOLUMAB, OBEXELIMAB, OBILTOXAXIMAB, OBINUTUZUMAB, OCARATUZUMAB, ODULIMOMAB, OFATUMUMAB, OLECLUMAB, OLENDALIZUMAB, OLINVACIMAB, OLOKIZUMAB, OMALIZUMAB, OMBURTAMAB, ONARTUZUMAB, ONTAMALIMAB, ONTUXIZUMAB, ONVATILIMAB, OPICINUMAB, OREGOVOMAB, ORILANOLIMAB, ORTICUMAB, OSOCIMAB, OTELIXIZUMAB, OTILIMAB, OTLERTUZUMAB, OXELUMAB, OZANEZUMAB, OZORALIZUMAB, PAGIBAXIMAB, PALIVIZUMAB, PAMREVLUMAB, PANITUMUMAB, PANOBACUMAB, PARSATUZUMAB, PASCOLIZUMAB, PASOTUXIZUMAB, PATECLIZUMAB, PATRITUMAB, PEMBROLIZUMAB, PEPINEMAB, PERAKIZUMAB, PERTUZUMAB, PEXELIZUMAB, PIDILIZUMAB, PINATUZUMAB, PLACULUMAB, PLAMOTAMAB, PLOZALIZUMAB, POLATUZUMAB, PONEZUMAB, PORGAVIXIMAB, POZELIMAB, PRASINEZUMAB, PREZALUMAB, PRILIXIMAB, PRITOXAXIMAB, PRITUMUMAB, PROLGOLIMAB, QUETMOLIMAB, QUILIZUMAB, RACOTUMOMAB, RADRETUMAB, RAFIVIRUMAB, RALPANCIZUMAB, RANEVETMAB, RANIBIZUMAB, RAVAGALIMAB, RAXIBACUMAB, REFANEZUMAB, REGAVIRUMAB, RELATLIMAB, RELFOVETMAB, REMTOLUMAB, RESLIZUMAB, RILOTUMUMAB, RINUCUMAB, RITUXIMAB, RIVABAZUMAB, ROBATUMUMAB, ROLINSATAMAB, ROMILKIMAB, RONTALIZUMAB, ROSMANTUZUMAB, ROVALPITUZUMAB, ROZANOLIXIZUMAB, SACITUZUMAB, SAMALIZUMAB, SAMROTAMAB, SARILUMAB, SATRALIZUMAB, SATUMOMAB, SECUKINUMAB, SELICRELUMAB, SEMORINEMAB, SERCLUTAMAB, SERIBANTUMAB, SETOXAXIMAB, SETRUSUMAB, SIBROTUZUMAB, SIFALIMUMAB, SIMTUZUMAB, SINTILIMAB, SIRTRATUMAB, SIRUKUMAB, SOFITUZUMAB, SOLANEZUMAB, SOLITOMAB, SONTUZUMAB, SPARTALIZUMAB, SPESOLIMAB, STAMULUMAB, SULESOMAB, SUPTAVUMAB, SUTIMLIMAB, SUVIZUMAB, SUVRATOXUMAB, TABALUMAB, TABITUXIMAB, TADOCIZUMAB, TAFASITAMAB, TALACOTUZUMAB, TALIZUMAB, TAMRINTAMAB, TAMTUVETMAB, TANEZUMAB, TAPLITUMOMAB, TAREXTUMAB, TAVOLIMAB, FANOLESOMAB, NOFETUMOMAB, PINTUMOMAB, TECLISTAMAB, TEFIBAZUMAB, TELIMOMAB, TELISOTUZUMAB, TEMELIMAB, TENATUMOMAB, TENELIXIMAB, TEPLIZUMAB, TEPODITAMAB, TEPROTUMUMAB, TESIDOLUMAB, TEZEPELUMAB, TIBULIZUMAB, TIDUTAMAB, TIGATUZUMAB, TILAVONEMAB, TILDRAKIZUMAB, TIMOLUMAB, TIRAGOLUMAB, TISLELIZUMAB, TISOTUMAB, TOCILIZUMAB, TOMARALIMAB, TORALIZUMAB, TORIPALIMAB, TOSATOXUMAB, TOSITUMOMAB, TOVETUMAB, TRALOKINUMAB, TRASTUZUMAB, TREGALIZUMAB, TREMELIMUMAB, TREVOGRUMAB, TUCOTUZUMAB, TUVIRUMAB, UBLITUXIMAB, ULOCUPLUMAB, URELUMAB, URTOXAZUMAB, USTEKINUMAB, UTOMILUMAB, VADASTUXIMAB, VANDORTUZUMAB, VANTICTUMAB, VANUCIZUMAB, VAPALIXIMAB, VARISACUMAB, VARLILUMAB, VATELIZUMAB, VELTUZUMAB, VEPALIMOMAB, VESENCUMAB, VIBECOTAMAB, VISILIZUMAB, VOBARILIZUMAB, VOFATAMAB, VOLAGIDEMAB, VOLOCIXIMAB, VONLEROLIZUMAB, VOPRATELIMAB, VORSETUZUMAB, VOTUMUMAB, VUNAKIZUMAB, XENTUZUMAB, ZALIFRELIMAB, ZAMPILIMAB, ZANOLIMUMAB, ZENOCUTUZUMAB, ZIRALIMUMAB, ZOLBETUXIMAB, and ZOLIMOMAB.

In the present disclosure, the term “idiotype” is a specialized concept of antibody engineering. Antibodies, as the most important effector molecules in the body's molecular recognition, have the characteristic of heterogeneity. The heterogeneity leads to isotypes, allotypes, and idiotypes. The concept of idiotype refers to the antigen specificity of an antibody molecule produced by each antibody-producing cell clone, which is determined by an amino acid sequence of a variable region of a light or the heavy chain of the antibody and thus is closely related to the antigen binding specificity of the antibody. The idiotype emphasizes the difference in the characteristic of antibody-antigen binding. A CAR also has an idiotype because the CAR recognizes an antigen based on its internal antibody structure.

In the present disclosure, a protein domain includes an antigen-binding domain, a hinge domain, a transmembrane domain, and an endodomain. The term “unique” means that a domain has different polypeptide (amino acid) sequences, includes different polypeptide (amino acid) sequences, or is composed of different polypeptide (amino acid) sequences. For example, two “unique” antigen-binding domains may bind to the same antigen (even the same epitope on the antigen), but the two antigen-binding domains are unique in that they have different consecutive amino acid compositions. Of course, two unique antigen-binding domains with different consecutive amino acid compositions can specifically bind to different antigens and epitopes.

A coding domain, domain, or gene may include any one or a combination of at least two selected from the group consisting of a protein-encoding amino acid sequence, a protein-encoding DNA sequence, or a protein-encoding RNA sequence.

The term “genetic circuit” used in the second genetic element is a concept of synthetic biology, and a genetic circuit is pre-programmed. In a broad sense, a genetic circuit includes a cis-regulatory factor and a transcription factor. The cis-regulatory factor may include a promoter, such as a T7 promoter, a CMV promoter, a UPS promoter, and a Tet promoter.

A complex regulatory network can be flexibly designed according to a research purpose, which is like a circuit network and thus is called genetic circuit.

In the present disclosure, the genetic circuit is composed of a cis-regulatory factor and/or a transcription factor.

The cis-regulatory factor may include a single cis-acting factor or a fusion cis-acting factor, and the transcription factor may include a single transcription factor or a combined transcription factor.

The single cis-acting factor may include any one or a combination of at least two selected from the group consisting of one or more NFAT-responsive promoter elements (NFAT), one or more NFκB-responsive promoter elements (NFκB), one or more tetracycline responsive elements (TRE), upstream activating sequences (UASs) of one or more galactose-metabolizing enzyme (GAL) gene promoters, one or more PIP responsive elements (PIR), one or more ZFHD1 responsive elements (ZFHD1RE), one or more ZF21-16 responsive elements (ZF21-16RE), one or more ZF42-10 responsive elements (ZF42-10RE), one or more ZF43-8 responsive elements (ZF43-8RE), one or more ZF54-8 responsive elements (ZF54-8RE), one or more minimal CMV promoters (P_{CMV-min}), one or more CMV promoters (P_{CMV}), one or more SV40 promoters (P_{SV40}), one or more minimal IL-2 promoters (P_{IL-2 min}), one or more minimal insect heat shock protein (HSP) 70 promoter (P_{hsp70 min}), and one or more minimal HIVtata promoters (P_{HIVtatamin}).

The fusion cis-acting factor may include a flexible combination of one or more single cis-acting factors, such as any one or a combination of at least two selected from the group consisting of a fusion of 4 NFAT-responsive elements and a minimal IL-2 promoter (4×NFAT), a fusion of 6 NFAT-responsive elements and a minimal IL-2 promoter (6×NFAT), a fusion of 5 NFκB-binding elements and a minimal HIVtata promoter (5×NFκB), a fusion of 10 NFκB-binding elements and a minimal HIVtata promoter (10×NFκB), a fusion of 7 TREs and a minimal CMV promoter (7×TRE-P_{CMV-min}), a fusion of 5 UASs and a minimal CMV promoter (5×UAS-P_{CMV-min}), a fusion of 4 PIRs and a minimal CMV promoter (4×PIR-P_{CMV-min}), a fusion of 8 PIRs and a minimal CMV promoter (8×PIR-P_{CMV-min}), a fusion of 8 PIRs and an insect heat shock protein 70 promoter (8×PIR-P_{hsp70 min}), a fusion of 4 ZFHD1REs and a minimal CMV promoter (4×ZFHD1RE-P_{CMV-min}), a fusion of 8 ZF21-16REs and a minimal CMV promoter (8×ZF21-16RE-P_{CMV-min}), a fusion of 8 ZF42-10REs and a minimal CMV promoter (8×ZF42-10RE-P_{CMV-min}), a fusion of 8 ZF43-8REs and a minimal CMV promoter (8×ZZF43-8R-P_{CMV-min}), a fusion of 8 ZF54-8REs and a minimal CMV promoter (8×ZF54-8RE-P_{CMV-min}), a fusion of 7 TREs and an SV40 promoter (7×TRE-P_{SV40}), a fusion of 7 TREs and a CMV promoter (7×TRE-P_{cmv}), a fusion of 5 UASs and an SV40 promoter (5×UAS-P_{SV40}), a fusion of 4 PIRs and an SV40 promoter (4×PIR-P_{SV40}), a fusion of 8 PIRs and an SV40 promoter (8×PIR-P_{SV40}), a fusion of 4 ZFHD1REs and an SV40 promoter (4×ZFHD1RE-P_{SV40}), a fusion of 8 ZF21-16REs and an SV40 promoter (8×ZF21-16RE-P_{SV40}), a fusion of 8 ZF42-10REs and an SV40 promoter (8×ZF42-10RE-P_{SV40}), a fusion of 8 ZF43-8REs and an SV40 promoter (8×ZZF43-8RE-P_{SV40}), and a fusion of 8 ZF54-8REs and an SV40 promoter (8×ZF54-8RE-P_{SV40}).

The combined transcription factor may include any one or a combination of at least two selected from the group consisting of TetR-VP64 (tTA), Gal4-VP64, PIP-VP64, ZF21-16-VP64, ZF-42-10-VP64, ZF43-8-VP64, ZF54-8-VP64, ZFHD1-VP64, Gal4-KRAB, TetR-KRAB, PIP-KRAB, ZF21-16-KRAB, ZF-42-10-KRAB, ZF43-8-KRAB, ZF54-8-KRAB, and ZFHD1-KRAB; and may preferably include any one or a combination of at least two selected from the group consisting of TetR-VP64 (tTA), Gal4-VP64, Gal4-KRAB, and TetR-KRAB.

In some specific embodiments of the present disclosure, the genetic circuit may preferably be composed of (i) any one selected from the group consisting of a combination of Gal4-KRAB and 5×UAS-P_{SV40}, a combination of Gal4-VP64 and 5×UAS-P_{CMV-min}, a combination of TetR-VP64 and 7×TRE-P_{CMV-min}, a combination of TetR-KRAB and 7×TRE-P_{SV40}, and a combination of TetR-KRAB and 7×TRE-P_{cmv} and (ii) any one selected from the group consisting of 4×NFAT, 6×NFAT, 5×NFκB, and 10×NFκB.

In the present disclosure, a unique genetic circuit means that there are genetic circuits resulting from different design and programming schemes. For example, two unique genetic circuits can achieve exactly the same biological effect (such as controlling the up-regulation of the expression of a downstream gene or controlling the down-regulation of the expression of a downstream gene), but the two genetic circuits are unique because a plurality of internal cis-acting factors and a plurality of regulatory genes (which can be transcription factors) therein are designed and constructed differently from each other. As used herein, the plurality of cis-acting factors and regulatory genes (which can be transcription factors) in the same genetic circuit are designed and constructed by the same scheme, and mutations merely within a range of homologous sequences do not affect the functions of the cis-acting factors and regulatory genes. The genetic circuits can be constructed by methods described in the following published documents: Kulemzin S V, et al. BMC Medical Genomics, 2019, 12(S2).; Uchibori R, et al. Molecular Therapy-Oncolytics, 2019, 12:16-25; Morsut L, et al. Cell, 2016, 164 (4): 780-791.; Deuschle U, Meyer W K, Thiesen H J. Molecular and Cellular Biology, 1995, 15 (4): 1907-1914.; Yang Zijie, et al. Chinese Journal of Biotechnology, 2018, 34 (12): 1886-1894.; Fussenegger M, et al. Nature Biotechnology, 2000, 18 (11): 1203-1208; Pomerantz J, Sharp P, Pabo C. Science, 1995, 267 (5194): 93-96.; Zhu Kaichuan, et al. Chinese Journal of Biotechnology, 2011, 31 (1): 81-85.; and Gene Transfer and Expression in Mammalian Cells. S. C. Makrides ELSEVIER 2003, which will not be repeated here.

The term “inducible protein” used in the third genetic element means that when the protein is expressed, a cell carrying the protein can be killed through induction; and the inducible protein may include a drug resistance protein and/or a suicide protein. The drug resistance protein refers to a protein resistant to a drug, and is a common technology in molecular biology and genetic engineering. The suicide protein is a technology emerging in recent years, where a genetic engineering process is often used to encode a prodrug hydrolase or induce an apoptosis signal to achieve the drug-induced death of a cell carrying the suicide protein.

Preferably, the drug resistance protein may include any one or a combination of at least two selected from the group consisting of a puromycin resistance protein, a neomycin resistance protein, a blasticidin resistance protein, and a hygromycin B resistance protein; and the suicide protein may include any one or a combination of at least two selected from the group consisting of a herpes simplex virus (HSV) thymidine kinase (TK) protein, a cytosine deaminase (CD) protein, and an inducible caspase 9 (iCasp9) suicide system protein.

An induction method of the inducible protein may include any one or a combination of at least two selected from the group consisting of drug induction, chemical induction, physical induction, laser induction, and thermal induction.

In the genetic element combination of the present disclosure, a plurality of first genetic elements may encode a plurality of idiotype CARs, a plurality of second genetic elements may be constructed into a plurality of unique genetic circuits, and a plurality of third genetic elements may encode a plurality of unique inducible proteins. In some other embodiments, a plurality of first genetic elements may encode a plurality of idiotype CARs, a plurality of second genetic elements may be constructed into a unique genetic circuit, and a plurality of third genetic elements may encode a plurality of unique inducible proteins. In some other embodiments, a plurality of first genetic elements may encode a plurality of idiotype CARs, a plurality of second genetic elements may be constructed into a plurality of unique genetic circuits, and a plurality of third genetic elements may encode a unique inducible protein.

Accordingly, the present disclosure relates to a genetic element combination library, namely, a library including different genetic element combinations; and the library is randomized with respect to idiotype CARs, genetic circuits, and inducible proteins, where structures of CARs may also be randomized with respect to unique antigen-binding domains (namely, antibody idiotypes), hinge domains, and/or endodomains, as shown in the patent document WO2015/123642.

Of course, the library can also be randomized with respect to the three genetic elements. For example, the library can be randomized with respect to idiotype CARs, genetic circuits, and inducible proteins; can also be randomized with respect to idiotype CARs and genetic circuits; can also be randomized with respect to idiotype CARs and inducible proteins; and can also be randomized with respect to unique genetic circuits and inducible proteins.

In a second aspect of the present disclosure, a construction method of a CAR cell library is provided.

In short, a first element, a second element, and a third element are inserted into one or more vectors, and then the vectors are transfected into cells to obtain the CAR cell library.

The vectors are well known to those skilled in the art, such as a viral vector described in the non-patent literature (Morsut L, et al. Cell, 2016, 164 (4): 780-791.) and a non-viral vector in the non-patent literature (Athanasopoulos T, et al. Hematology/Oncology Clinics of North America, 2017, 31 (5): 753-770.). The genetic elements can also be inserted into the vectors at a specific site such as AAVS1 site (Parthiban K, et al. mAbs. Taylor & Francis, 2019.).

A method for the transfection may include any one or a combination of at least two selected from the group consisting of viral transfection, chemical transfection, and electroporation transfection.

The cells may be mammalian cells, and may preferably immune cells, including immune cells and/or genetically-engineered immune cells. The immune cells may include any one or a combination of at least two selected from the group consisting of autologous immune cells, donor-derived immune cells, and healthy volunteer-derived immune cells. The immune cells may be preferably T lymphocytes and more preferably NK cells such as NK-92 cells.

The construction method specifically includes the following steps:

A. Preparation of an Antibody Display Library

An antibody gene library is established through a healthy volunteer source or a total synthesis process, and a functional gene library can also be established through a genetic engineering process. With the genetic engineering process as an example, a suitable display platform is selected based on the healthy volunteer source or the total synthesis process to establish an antibody display library, then the background is deducted through multiple rounds of panning with a control tissue as a control to obtain a phage antibody display sub-library, and then the polymerase chain reaction (PCR) is conducted to obtain an antibody gene library.

B. Construction of Genetic Elements

A first genetic element including a scFV library-CAR is constructed, where the scFV library is constructed as the extracellular recognition domain of CAR. A second genetic element including a first cis-regulatory factor, a transcription factor regulated by the first cis-regulatory factor, and a second cis-acting factor regulated by the transcription factor is constructed. A third genetic element including an inducible protein gene is constructed. The three genetic elements are inserted into one or more controlled gene expression cassettes.

C. Introduction of the Genetic Elements into the Cells

The one or more controlled gene expression cassettes are introduced into mammalian immune cells through a lentiviral vector system to obtain the CAR cell library.

In a third aspect of the present disclosure, a CAR cell library is provided.

The CAR cell library carries the genetic element combination according to the first aspect and is constructed by the construction method according to the second aspect.

In a fourth aspect of the present disclosure, a method for screening a CAR cell targeting an in vitro antigen is provided, including the following steps:

(1) The CAR cell library is allowed to contact the antigen. (2) CAR cells are screened according to the expression of a suicide protein. (3) Target CAR cells are enriched.

Preferably, the method may further include the following steps:

(4) A secondary CAR cell library is reconstructed through an antibody engineering process from the target CAR cells obtained in step (3). (5) Steps (1) to (3) are repeated, and when the target CAR cells are screened, steps (4) and (5) are repeated one or more times if necessary.

The CAR cells may include monoclonal CAR cells and polyclonal CAR cell populations.

The antigen may include any one or a combination of at least two selected from the group consisting of a wild-type (WT) cell, a cell transfected with a specific antigen gene, a cell binding to a specific antigen, an antigen dissolved in a medium, an antigen coated on a petri dish, an antigen coated on a microbead, and an antigen coated on a culture scaffold.

In the present disclosure, the method for screening a CAR cell is as follows: the CAR cell library is allowed to contact the in vitro antigen, and only in the CAR cell capable of recognizing the antigen, the inducible protein can undergo any one or a combination of at least two selected from the group of the following conditions according to the pre-programmed genetic circuit in the cell: activating the expression of the inducible protein in the cell, enhancing the expression of the inducible protein in the cell, terminating the expression of the inducible protein in the cell, and inhibiting the expression of the inducible protein in the cell. The screening method is determined according to the expression of the inducible protein, and then the target CAR cell is screened out. The screening method can be flexibly designed according to the genetic circuit and inducible protein to achieve the screening purpose.

In some specific embodiments of the present disclosure, the following screening method may be adopted: the CAR-responsive genetic circuit is designed to activate and/or enhance the expression of the inducible protein when the CAR is activated, and the inducible protein is designed as a suicide protein; and thus in a cultivation environment with a screening drug, CAR-activated cells undergo apoptosis and the remaining cells survive, such that a CAR cell that cannot bind to the target antigen is screened out.

In some specific embodiments of the present disclosure, the following screening method may be adopted: the CAR-responsive genetic circuit is designed to terminate and/or inhibit the expression of the inducible protein when the CAR is activated, and the inducible protein is designed as a suicide protein; and thus in a cultivation environment with a screening drug, CAR-activated cells survive and the remaining cells undergo apoptosis, such that a CAR cell that can bind to the target antigen is screened out.

In some specific embodiments of the present disclosure, the following screening method may be adopted: the CAR-responsive genetic circuit is designed to activate and/or enhance the expression of the inducible protein when the CAR is activated, and the inducible protein is designed as a drug resistance protein; and thus in a cultivation environment with a screening drug, CAR-activated cells survive and the remaining cells undergo apoptosis, such that a CAR cell that can bind to the target antigen is screened out.

In some specific embodiments of the present disclosure, the following screening method may be adopted: the CAR-responsive genetic circuit is designed to terminate and/or inhibit the expression of the inducible protein when the CAR is activated, and the inducible protein is designed as a drug resistance protein; and thus in a cultivation environment with a screening drug, CAR-activated cells undergo apoptosis and the remaining cells survive, such that a CAR cell that cannot bind to the target antigen is screened out.

In some specific embodiments of the present disclosure, the following screening method may also be included: any one or a combination of at least two selected from the group consisting of drug screening, flow cytometry (FCM) assay and sorting, magnetic bead sorting, and bead sorting.

Preferably, the screening drug for the drug resistance protein may include any one selected from the group consisting of puromycin, G418, blasticidin, and hygromycin B; and the screening drug for the suicide protein may include any one or a combination of at least two selected from the group consisting of ganciclovir or FIAU, 5-flucytosine, AP1903, and AP20187.

In a fifth aspect of the present disclosure, a method for screening a CAR cell targeting an in vivo antigen is provided. The method includes the following steps: (1) An effective amount of the CAR cell library is administered to an experimental subject. (2) The screening is conducted according to the expression of the suicide protein. (3) The target CAR cell is enriched and enriched by a general method from the subject.

Preferably, the method may further include the following steps: (4) A secondary CAR cell library is reconstructed through an antibody engineering and/or a genetic engineering process from the target CAR cells obtained in step (3). (5) Steps (1) to (3) are repeated, and when the target CAR cells are screened, steps (4) and (5) are repeated one or more times if necessary.

The CAR cells may include monoclonal CAR cells and polyclonal CAR cell populations.

The in vivo antigen may refer to an antigen existing in a living human or animal, and may include any one or a combination of at least two selected from the group consisting of an in vivo cell, an in vivo lesion cell, an in vivo cell transfected with a specific antigen gene, an in vivo cell infected with a specific pathogen, an in vivo cell binding to a specific antigen, and an in vivo cell transplanted into an animal model.

A method for allowing the CAR cell library to contact the in vivo antigen may generally include in vivo administration of the CAR cell library. An administration route may include any one or a combination of at least two selected from the group consisting of intravenous infusion, gastrointestinal infusion, intramuscular injection, local tissue injection, subcutaneous injection, intraperitoneal injection, and inhalation.

In the present disclosure, the method for screening a CAR cell is as follows: the CAR cell library is administered to the subject of interest such that the CAR cell library contacts the in vivo antigen, and only in the CAR cell capable of recognizing the antigen, the inducible protein can undergo any one or a combination of at least two selected from the group of the following conditions according to the pre-programmed genetic circuit in the cell: activating the expression of the inducible protein in the cell, enhancing the expression of the inducible protein in the cell, terminating the expression of the inducible protein in the cell, and inhibiting the expression of the inducible protein in the cell. The screening method is determined according to the expression of the inducible protein, and then the target CAR cell is screened out. The screening method can be flexibly designed according to the genetic circuit and inducible protein to achieve the screening purpose. A specific screening method is the same as above.

In a sixth aspect of the present disclosure, a CAR cell is provided. The CAR cell is screened by the methods according to the fourth aspect and the fifth aspect. The CAR cell may include a monoclonal CAR cell and a polyclonal CAR cell.

In a seventh aspect of the present disclosure, a CAR is provided. The CAR is obtained by extracting a relevant gene from the CAR cell according to the sixth aspect through a general genetic engineering technology.

In an eighth aspect of the present disclosure, an antibody is provided. The antibody is obtained by a general antibody engineering technology based on the CAR according to the seventh aspect.

In a ninth aspect of the present disclosure, a target antigen is provided. The target antigen is obtained by a general genetic engineering technology based on the CAR cell according to the sixth aspect, the CAR according to the seventh aspect, and the antibody according to the eighth aspect.

In a tenth aspect of the present disclosure, a pharmaceutical composition is provided. The pharmaceutical composition includes any one or a combination of at least two selected from the group consisting of the CAR cell library according to the first aspect, a CAR cell library constructed by the construction method according to the second aspect, the CAR cell according to the sixth aspect, the CAR according to the seventh aspect, and the antibody according to the eighth aspect, and at least one medically/pharmaceutically acceptable carrier. In addition, the pharmaceutical composition may further include a medical/pharmaceutical diluent or excipient.

In an eleventh aspect of the present disclosure, a use of the pharmaceutical composition in the preparation of any one or a combination of at least two selected from the group consisting of a drug, a reagent, and a kit for the prevention, diagnosis, and treatment of a disease that requires the removal of a disease-associated mediator is provided.

In some aspects, the disease-associated mediator refers to lesion cells, and diseases whose treatment requires the removal of lesion cells may include benign and malignant tumors, tissue hyperplasia, acute and chronic inflammation, or the like. The diseases can be, for example, malignant tumors of lung, breast, stomach, pancreas, prostate, bladder, bone, ovary and adnexa, uterus, skin, kidney, sinus, colon, rectum, esophagus, blood, brain and covering thereof, spinal cord and covering thereof, muscle, connective tissue, adrenal, parathyroid, thyroid, testis, pituitary, genitals, liver, gallbladder, eyes, ears, nose, larynx, tonsils, mouth, lymph nodes and lymphatic system, and other organs. In some embodiments, benign tumors and acute and chronic inflammation of these organs may also be included.

In the present disclosure, the term “malignant tumors” may include all forms of human carcinomas, sarcomas, and melanomas, and these forms may include poorly-differentiated, moderately-differentiated, and highly-differentiated forms.

The diseases may further include hyperplasia, hypertrophy, ectopia, or excessive growth of tissues, and the tissues may include lung, breast, stomach, pancreas, prostate, bladder, bone, ovary and adnexa, uterus, skin, kidney, sinus, colon, rectum, esophagus, blood, brain and covering thereof, spinal cord and covering thereof, muscle, connective tissue, adrenal, parathyroid, thyroid, testis, pituitary, genitals, liver, gallbladder, eyes, ears, nose, larynx, tonsils, mouth, lymph nodes and lymphatic system, and other organs.

The diseases may further include viral, bacterial, or parasitic changes of tissues, and the tissues may include lung, breast, stomach, pancreas, prostate, bladder, bone, ovary and adnexa, uterus, skin, kidney, sinus, colon, rectum, esophagus, blood, brain and covering thereof, spinal cord and covering thereof, muscle, connective tissue, adrenal, parathyroid, thyroid, testis, pituitary, genitals, liver, gallbladder, eyes, ears, nose, larynx, tonsils, mouth, lymph nodes and lymphatic system, and other organs.

The diseases may further include fibrosis changes of tissues caused by acute and chronic inflammation, and the tissues may include lung, breast, stomach, pancreas, prostate, bladder, bone, ovary and adnexa, uterus, skin, kidney, sinus, colon, rectum, esophagus, blood, brain and covering thereof, spinal cord and covering thereof, muscle, connective tissue, adrenal, parathyroid, thyroid, testis, pituitary, genitals, liver, gallbladder, eyes, ears, nose, larynx, tonsils, mouth, lymph nodes and lymphatic system, and other organs.

The diseases may further include endometriosis, tonsillar hypertrophy, prostatic hyperplasia, psoriasis, eczema, skin diseases, hemorrhoids, and vascular diseases such as atherosclerosis, arteriosclerosis, varicose veins, or stenosis or severe stenosis of arteries or stents.

The diseases may further include cosmetic repair and anti-aging of tissues, such as cosmetic repair of skin, eyes, ears, nose, larynx, mouth, muscle, connective tissue, hair, or breast tissue.

The diseases may further include neurodegenerative diseases such as Alzheimer's disease and Parkinson's disease.

In some aspects, the disease-associated mediator may refer to an inflammatory mediator.

In some methods, the inflammatory mediator may include any one or a combination of at least two selected from the group consisting of pathogens such as viruses, bacteria, or parasites, enzymes, cytokines, prostaglandins, eicosanoids, leukotrienes, kinins, complements, coagulation factors, toxins, endotoxins, enterotoxins, lipopolysaccharides, apoptosis-inducing substances, corrosive substances, bile salts, fatty acids, phospholipids, oxidation by-products, reactive oxygen species (ROS), oxygen free radicals, surfactants, ions, stimulating substances, cell debris, interferons, immunomodulatory antibodies, biologics, and drugs. In some aspects, the inflammatory mediator may exist in a physiological fluid or carrier fluid of a subject; and the physiological fluid may include a nasopharyngeal fluid, an oral fluid, an esophageal fluid, a gastric fluid, a pancreatic fluid, a liver fluid, a pleural fluid, a pericardial fluid, a peritoneal fluid, an intestinal juice, a prostatic fluid, a seminal fluid, a vaginal secretion, tears, saliva, mucus, bile, blood, lymph, plasma, serum, a synovial fluid, a cerebrospinal fluid, an uterine and adnexal fluid, urine, and an intercellular, intracellular, or extracellular fluid.

Inflammatory mediator-associated diseases may include: systemic inflammatory response syndrome (SIRS) or sepsis (caused by viral, bacterial, fungal, or parasitic infection, for example), autoimmune diseases, diseases resulting from surgery, cytotoxic chemotherapy, and bone marrow transplantation, major tissue damage or trauma, mesenteric hypoperfusion, intestinal mucosal injury, malaria, gastrointestinal inflammatory diseases, intestinal infection, intrauterine infection, influenza, acute pneumonia such as acute respiratory distress syndrome (ARDS) or acute lung injury, pulmonary embolism, pancreatitis, autoimmune and collagen vascular disease, transfusion-associated diseases, burns, smoke inhalation lung injury, graft-versus-host disease (GVHD), ischemia or infarction, reperfusion injury, hemorrhage, anaphylaxis, drug overdose, radiation injury, or chemical damage. In some embodiments, the inflammatory mediator may be produced in diseases caused by pathogens, toxins, or agents of biological warfare, such as viral hemorrhagic fevers (VHFs), marine toxins such as jellyfish toxins, dengue fever, Ebola, Hantavirus cardiopulmonary syndrome (HCPS) (Hantavirus), cholera toxin, botulinum toxin, ricin toxin, Q fever (Coxiella burnetii), typhus (Rickettsia prowazekii), or psittacosis (Chlamydia psittaci).

The inflammatory mediator-associated diseases may further include diseases whose treatment requires the removal of a target immune factor, such as transplantation and immune infertility.

Compared with the prior art, the present disclosure has the following technical effects:

Unique and creative features of the present disclosure: (1) The present disclosure solves the scientific problem that disease antigens are unknown and have variability, evolvability, and heterogeneity. The CAR library in the present disclosure carries three genetic elements and realizes a cascade reaction of CAR-genetic circuit-inducible protein, where when the CAR encoded by the first genetic element is activated, the genetic circuit pre-programmed in the second genetic element plays an expression regulation role for the inducible protein encoded by the third genetic element, such as activating transcription and expression, enhancing transcription and expression, terminating transcription and expression, and inhibiting transcription and expression. Since the genetic circuit is pre-programmed, the CAR cell library of the present disclosure can achieve programmatic changes in response to antigen changes in vivo, and is especially suitable for acquiring cell libraries targeting individualized antigens. That is, the present disclosure proposes a brand-new cell library that can change with a disease antigen, such as to realize the preparation of an individualized product. However, CAR libraries in the prior art cannot respond to variable antigens. (2) The CAR cell library of the present disclosure also has an advantage that a pharmacodynamic index can be directly added during the screening to achieve the purpose of directly preparing a therapeutic product.

In addition, the present disclosure also provides a construction method and a screening method of the CAR cell library. The construction method essentially includes the construction of a vector and the transfection of the vector into cells, which is mature and easy to master and helps to reduce the cost of library construction and screening.

Therefore, since the CAR cell library of the present disclosure is based on a CAR library, a CAR library-genetic circuit-inducible protein coupling scheme is designed to realize the cell library construction and screening for complex and unknown disease target antigens. The cell library and the construction and screening method thereof can solve the problems that there are complex, diverse, and variable antigens and it is difficult to identify a target in aberrant antigen-expressing diseases, which has promising application prospects.

### BRIEF DESCRIPTION OF THE DRAWINGS

FIG. 1 is a schematic diagram illustrating a structure, a construction method, and in vivo screening of the CAR cell library according to an embodiment of the present disclosure, where A shows an antibody library constructed by an existing technology; B shows a background-subtracted antibody library; C shows the construction of the CAR cell library; D shows the in vitro screening; E shows the in vivo screening; F shows the clearance of CAR cells failing to identify a target antigen; and G shows the survival of the CAR cells recognizing the target antigen.

FIG. 2 is a schematic diagram of controlled gene expression cassettes, where A is a schematic structural diagram of the controlled gene expression cassette NFAT-KRAB-iCasp9-2A-GFP; B is a schematic structural diagram of the controlled gene expression cassette CMV-scFvlab-CAR; and C is a schematic structural diagram of the controlled gene expression cassette CD19-CAR.

FIG. 3 shows the tumor volume change in each group after mouse tumor models are treated with the cell library.

FIG. 4 shows the expression of CAR in blood cells of animals in each group after treatment.

FIG. 5 shows the tumor volume change in each group after mouse tumor models are treated with the screened cells.

FIG. 6 shows the tumor tissue inhibition rate of each group after PDX models are treated with the cell library.

FIG. 7 shows the pathological score of each group with inflammatory bowel disease (IBD).

FIG. 8 is a schematic diagram of the construction of genetic elements, where A to F show the construction of the first genetic element and G to K show the construction of the second genetic element.

FIG. 9 is a schematic diagram illustrating the assembly of a vector.

FIG. 10 is a schematic diagram illustrating individualized genetic elements and vectors.

### DETAILED DESCRIPTION

The following examples and experimental examples are provided to further illustrate the present disclosure, and shall be construed as a limitation to the present disclosure. The examples do not include detailed descriptions of traditional methods, such as common antibody engineering methods, methods for constructing vectors and plasmids, methods for inserting genes encoding proteins into such vectors and plasmids, methods for introducing plasmids into host cells, and construction methods of synthetic cells, devices, and genetic circuits. Such methods are well known to those of ordinary skill in the art, and are described in many publications, including Antibody Engineering (2nd edition) edited by Dong Zhiwei and Wang Yan, Peking University Medical Press (PUMP), 2002; Sambrook, J., Fritsch, E. F. and Maniais, T. (1989) Molecular Cloning: A Laboratory Manual, 2nd edition, Cold spring Harbor Laboratory Press; and Phage Display: A laboratory Manual, Cold spring Harbor Laboratory Press.

### EXAMPLE 1

### Totally-Synthetic Mouse-Derived CAR-T Cell Library

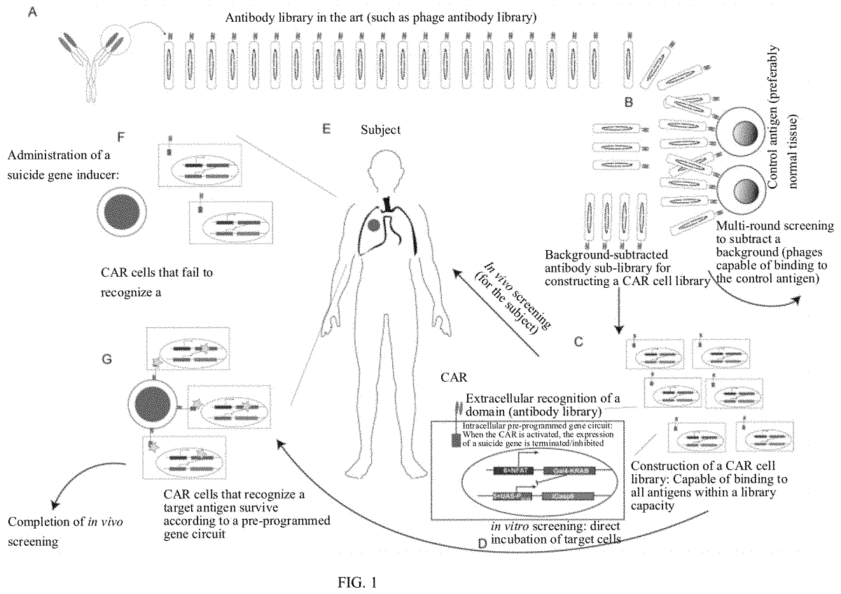

A construction and screening process of the library was shown in FIG. 1:

(A) Construction of a phage antibody library: A totally-synthetic mouse-derived phage scFV library was constructed through total synthesis. A method for constructing the antibody library is well known to those of ordinary skill in the art, and a method for constructing the totally-synthetic mouse-derived phage scFV library is the same as in the literature (Geuijen C et al. European Journal of Cancer, 2005, 41 (1): 178-187; Noronha E J, et al. Journal of Immunology, 1998, 161 (6): 2968-2976.). According to library capacity evaluation, the totally-synthetic mouse-derived phage scFV library had a library capacity of 1×10⁹. A method of the library capacity evaluation is the same as in the literature (Ridgway J B B, et al. Cancer Research, 2013, 59 (11): 2718-2723).

(B) Background removal: The totally-synthetic mouse-derived phage scFV library (1×10¹¹ PFU) was injected into BALB/c mice through the tail vein, and 4 rounds of screening were conducted to remove phage capable of binding to mouse tissues to obtain a phage antibody sub-library. A method of the screening is the same as in the literature (Wada, Akinori, et al. Molecular Therapy-Oncolytics 12 (2019): 138-146.). The sub-library was subjected to amplification detection, and it was found that the library capacity did not change significantly. Then an antibody gene library was obtained by a PCR method.

(C) Construction of a CAR Cell Library

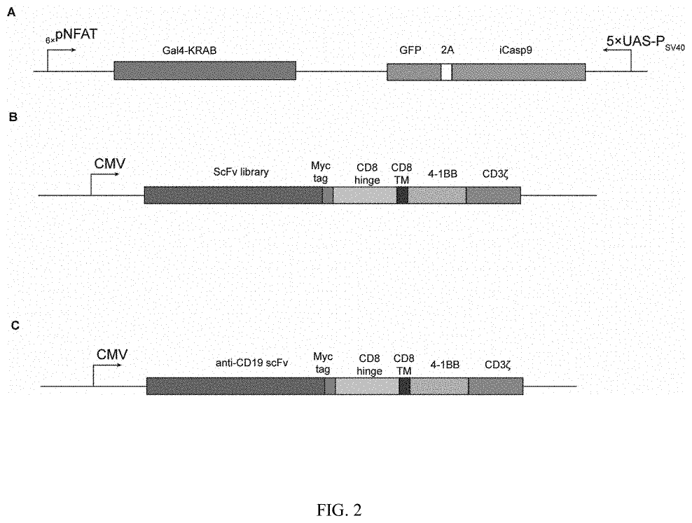

Mouse T lymphocytes were acquired according to the method reported in the literature [Srivastava S, et al. Cancer cell, 2019, 35 (3): 489-503. e8.]. Then a genetic circuit was constructed (FIG. 2). The genetic circuit involved two controlled gene expression cassettes: (i) Controlled gene expression cassette NFAT-KRAB-iCasp9-2A-GFP shown in FIG. 2A: The cassette included an NFAT-responsive element promoter (Uchibori R, et al. Molecular Therapy-Oncolytics, 2019, 12: 16-25.), a fusion of 6 NFAT-responsive elements and a minimal IL-2 promoter that controlled the expression of the repressive transcription factor GAL4-KRAB (Morsut L, et al. Cell, 2016, 164 (4): 780-791.), and a 5×UAS-PSV40 promoter fusion (Morsut L, et al. Cell, 2016, 164 (4): 780-791.) that controlled the iCasp9-2A-green fluorescent protein fusion gene. Construction methods of the iCasp9 gene and self-cleaving peptide 2A are the same as in the literature [Liu E, et al. Leukemia, 2018, 32 (2): 520.]. The controlled gene expression cassette NFAT-KRAB-iCasp9-2A-GFP was integrated into the mouse T lymphocytes through a lentiviral vector system. Mouse T lymphocytes successfully introduced with the cassette were sorted by FCM. ii) Controlled gene expression cassette CMV-scFvlab-CAR shown in FIG. 2B: The above mouse-derived scFV library was constructed as an extracellular recognition domain of CAR. The mammalian cell gene integration technology reported in the literature [Parthiban K, et al. mAbs. Taylor & Francis, 2019.] was used to integrate the controlled gene expression cassette CMV-scFvlab-CAR into T cells through a lentiviral vector system.

The genetic circuit was pre-programmed as follows: when the CAR bound to an antigen, the expression of the iCasp9 gene was inhibited under a change of the genetic circuit, and a corresponding cell did not undergo the induced apoptosis regulation by an iCasp9 inducer.

The obtained CAR cell library was named KRAB-iCasp9-CAR-T, which had a library capacity of 1×10⁶.

(D) In vitro screening: The cell library was directly incubated with a target antigen.

(E) In vivo screening: A solution of the cell library was administered to a subject at an appropriate amount and concentration.

(F) A suicide gene iCasp9 inducer was administered, the inhibition on the expression of the iCasp9 gene was detected according to the pre-programmed genetic circuit to screen out cells undergoing the induced apoptosis regulation of the iCasp9 inducer, and the cells were determined as CAR cells failing to recognize the target antigen and cleared.

(G) According to the inhibition of the expression of the iCasp9 gene in vivo, cells free from the induced apoptosis regulation of the iCasp9 inducer were screened out and enriched.

### EXAMPLE 2

### Treatment of Breast Cancer with the Totally-Synthetic Mouse-Derived CAR-T Cell Library

This example was implemented with the KRAB-iCasp9-CAR-T cell library obtained in Example 1.

4T1 mouse breast cancer in situ models were constructed by a method in the literature (Paschall A V, Liu K. JoVE 2016 (114): e54040.). When an average tumor volume in mice reached 100 mm³, the mice were divided into a control group, an irrelevant CAR-T cell group, a KRAB-iCasp9-CAR-T cell library group 1, a KRAB-iCasp9-CAR-T cell library group 2, a KRAB-iCasp9-CAR-T cell library group 3, and a KRAB-iCasp9-CAR-T cell library group 4.

A CAR-positive rate of CAR-T cells was normalized to 45%. The control group was administered with PBS; the irrelevant CAR-T cell group was administered with CD19-CAR-T cells (the controlled gene expression cassette shown in FIG. 2C), and the cells were intravenously injected at a dose of 5×10⁶ cells once every 2 d, 3 times in total; and all KRAB-iCasp9-CAR-T cell library groups were each administered with the KRAB-iCasp9-CAR-T cell library, and the cells were diluted with the serum-free 1640 medium and then intravenously injected at a dose of 5×10⁶ cells once every 2 d, 3 times in total. The KRAB-iCasp9-CAR-T cell library group 1 was only subjected to cell therapy; the KRAB-iCasp9-CAR-T cell library group 2 was administered with an iCasp9 inducer from the beginning of the therapy, with a dose and route the same as in the literature [Liu E, et al. Leukemia, 2018, 32 (2): 520.]; the KRAB-iCasp9-CAR-T cell library group 3 was administered with an iCasp9 inducer from the second week of the therapy, with a dose and route the same as above; and the KRAB-iCasp9-CAR-T cell library group 4 was administered with an iCasp9 inducer from the second week of the therapy, but the iCasp9 inducer was discontinued immediately after tumor regression, and the experiment of the group was stopped accordingly.

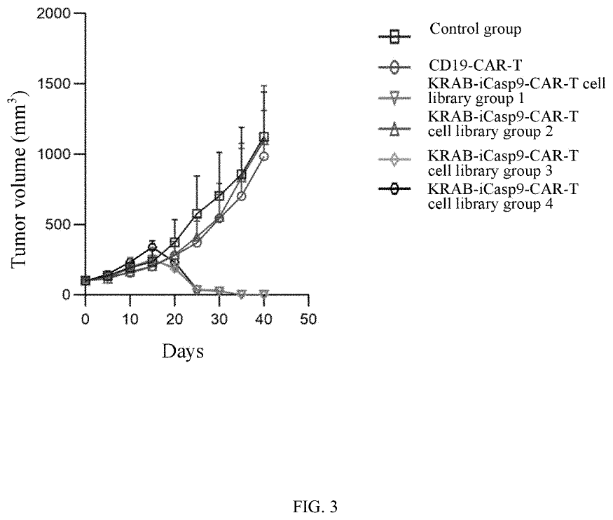

Experimental results were shown in FIG. 3, and it can be seen that, in the control group and irrelevant CAR-T cell group, the tumor tissue volume increased rapidly; in the KRAB-iCasp9-CAR-T cell library group 1, KRAB-iCasp9-CAR-T cell library group 3, and KRAB-iCasp9-CAR-T cell library group 4, the tumor growth was significantly inhibited from the second week of the therapy, and the tumor was rapidly cleared; and in the KRAB-iCasp9-CAR-T cell library group 2 administered with the iCasp9 inducer from the beginning of the therapy, no tumor-inhibition effect was exhibited.

### EXAMPLE 3

### In Vivo Screening of Totally-Synthetic Mouse-Derived CAR-T Cells Targeting 4T1 Breast Cancer

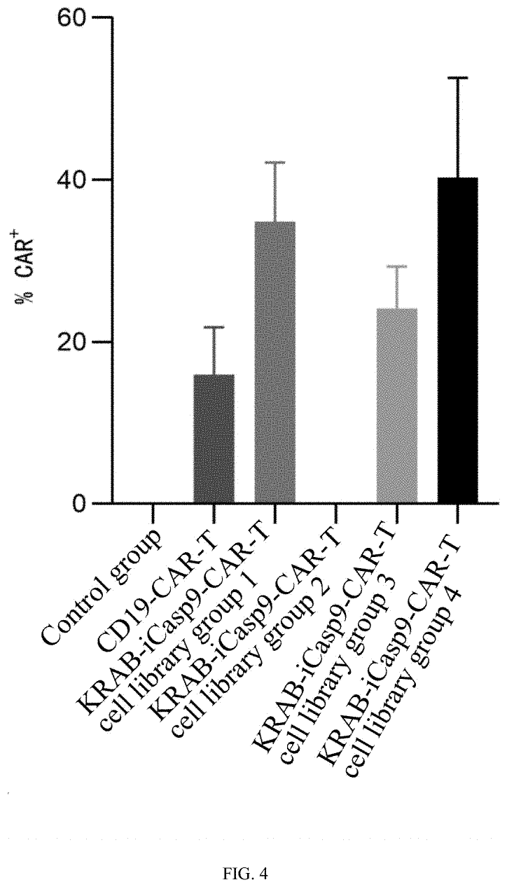

Mice in all experimental groups in Example 2 were sacrificed after the experiment, blood was collected from the mice, and CAR-positive cells in the blood were detected by FCM. Proportions of CAR-positive cells in all experimental groups obtained after the experiment were shown in FIG. 4. The CAR-positive cells in each group were isolated and cultivated to complete the in vivo screening.

### EXAMPLE 4

### In Vitro Screening of Totally-Synthetic Mouse-Derived CAR-T Cells Targeting 4T1 Breast Cancer

This example was implemented with the KRAB-iCasp9-CAR-T cell library obtained in Example 1. The KRAB-iCasp9-CAR-T cell library (1×10⁷ cells, CAR positive rate: 70%) was co-cultivated with 1×10⁷ of 4T1 breast cancer cells for 96 h, then an iCasp9 inducer was added to a medium with an amount and manner the same as in the literature (Liu E, et al. Leukemia, 2018, 32 (2): 520.), and 5 d later, CAR-positive cells were separated by FCM to complete the in vitro screening.

### EXAMPLE 5

### Preparation of a CAR and Antibody Targeting 4T1 Breast Cancer Cells

The T cell genome was extracted from the CAR-T cells screened out in Examples 3 and 4 with a kit. Primers were designed to acquire the CAR gene through PCR, that is, the CAR targeting 4T1 breast cancer cells was obtained. A scFV was further obtained through PCR, the obtained scFV was constructed as mouse IgG2a through genetic engineering, and then the antibody targeting 4T1 breast cancer cells was obtained through expression and purification.

### EXAMPLE 6

### Identification of a Target Antigen of 4T1 Breast Cancer Cells

The antibody obtained in Example 5 was cross-linked on agarose beads, then a 4T1 breast cancer cell lysate was incubated overnight with the antibody-cross-linked beads, and the beads were rinsed, such that an antigen targeted by the antibody was enriched on the beads; and the beads were subjected to peptide mass fingerprinting (PMF) identification to obtain the target antigen.

### EXAMPLE 7

### Therapeutic use of Totally-Synthetic Mouse-Derived CAR-T Cells Targeting 4T1 Breast Cancer

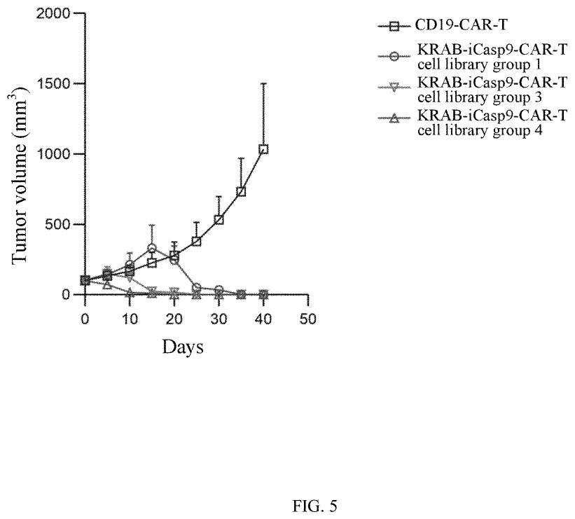

The CAR-T cells obtained in each group in Example 3 were used once again for the treatment of breast cancer in situ in 4T1 mice. A treatment method of each group was the same as above, and an iCasp9 inducer was administered from the second week of the treatment. Results were shown in FIG. 5. A very obvious anti-tumor effect was exhibited in the KRAB-iCasp9-CAR-T cell library group 3 and KRAB-iCasp9-CAR-T cell library group 4.

### EXAMPLE 8

### Natural Fully Human CAR NK-92 Cell Library

A natural human-derived phage scFV library was constructed with PBMCs of 200 healthy volunteers. A method for constructing the phage scFV library is the same as in the literature (Geuijen C et al. European Journal of Cancer, 2005, 41 (1): 178-187; Noronha E J, et al. Journal of Immunology, 1998, 161 (6): 2968-2976.). According to library capacity evaluation, the natural human-derived phage scFV library had a library capacity of 1×10¹⁰.

The natural human-derived phage scFV library (1×10¹²PFU) was injected into NSG mice through the tail vein, and 4 rounds of screening were conducted to remove phage capable of binding to mouse tissues. A phage antibody library obtained after the screening was subjected to amplification detection, and it was found that the library capacity did not change significantly. Then an antibody gene library was obtained by a PCR method.

NK-92 cells (Schonfeld K, et al. Molecular therapy, 2015, 23 (2): 330-338.) were adopted to further construct a cell library. CARs and intracellular genetic circuits were constructed according to the same method as in Example 1 to obtain the natural fully human CAR NK-92 cell library. The obtained natural fully human CAR NK-92 cell library was named KRAB-iCasp9-CAR-NK92, which had a library capacity of 1×10⁶.

### EXAMPLE 9

### In Vivo Screening of the Natural Fully Human CAR NK-92 Cell Library

This example was implemented with the KRAB-iCasp9-CAR-NK92 library obtained in Example 8.

NSG mouse tumor-bearing models (namely PDX) were directly constructed with patient-derived tissues. According to the method in the literature (Fu W, et al. Clinical Cancer Research, 2019, 25 (9): 2835-2847.), a lung cancer PDX model L10, a breast cancer PDX model B7, and an ovarian cancer PDX model OV3 were constructed. When an average tumor volume in mice reached 400 mm³, the mice were divided into a control group, an irrelevant CAR-NK92 cell group (anti-CD19 CAR-NK92, including the controlled gene expression cassette shown in FIG. 2C), and a KRAB-iCasp9-CAR-NK92 library group. The same therapeutic dose and manner as in Example 1 were adopted. At the second week of treatment, each group was administered with an iCasp9 inducer. Mice were sacrificed at the third week of treatment, blood and tumor tissues were collected from the mice, and CAR-positive cells were detected by FCM.

In each of the lung cancer PDX model L10, the breast cancer PDX model B7, and the ovarian cancer PDX model OV3, CAR-positive cells were isolated from the KRAB-iCasp9-CAR-NK92 library group to complete the in vivo screening.

### Example 10

### Construction of a CAR NK-92 Cell Library Targeting a Tumor Tissue

This example was implemented with the natural fully human CAR NK-92 cells obtained from the in vivo screening in Example 9.

CARs targeting lung cancer L10, breast cancer B7, and ovarian cancer OV3 were obtained from the natural fully human CAR NK-92 cells obtained from the in vivo screening in Example 9 through a general genetic engineering technology. Then PCR was used to obtain extracellular scFV sequences of the CARs. Random mutations of the heavy chain CDR3 were designed through computer-aided design (CAD) according to the extracellular scFV sequence, and a scFV library was re-constructed through artificial synthesis, which was a genetically-engineered antibody library targeting a tumor tissue.

NK-92 cells were adopted to further prepare a cell library, and CARs and intracellular genetic circuits were constructed according to the same method as in Example 1. The extracellular recognition domain of CAR was the genetically-engineered antibody library. Finally, CAR NK-92 cell libraries respectively targeting lung cancer L10, breast cancer B7, and ovarian cancer OV3 were obtained. The obtained human CAR NK-92 cell libraries were respectively named L10-KRAB-iCasp9-CAR-NK92, B7-KRAB-iCasp9-CAR-NK92, and OV3-KRAB-iCasp9-CAR-NK92, which each had a library capacity of 1×10⁵.

### EXAMPLE 11

### Treatment of Mouse Tumor-Bearing Models with the CAR NK-92 Cell Libraries Targeting Tumor Tissues

This example was implemented with the libraries L10-KRAB-iCasp9-CAR-NK92, B7-KRAB-iCasp9-CAR-NK92, and OV3-KRAB-iCasp9-CAR-NK92 obtained in Example 10.

A lung cancer PDX model L10, a breast cancer PDX model B7, and an ovarian cancer PDX model OV3 were constructed. When an average tumor volume in mice reached 100 mm³, the mice were divided into a control group, an irrelevant CAR-NK92 cell group (anti-CD19 CAR-NK92, including the controlled gene expression cassette shown in FIG. 2C), and NK-92 cell library groups. The same therapeutic dose and manner as in Example 1 were adopted.

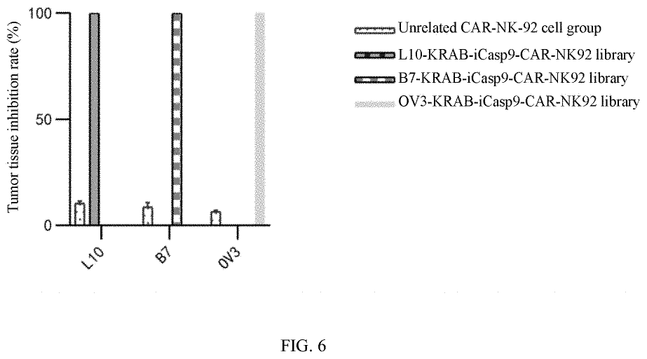

At the second week of treatment, each group was administered with an iCasp9 inducer. After 5 weeks of treatment, a tumor growth inhibition ratio was calculated as follows: ratio=1−average tumor volume in treatment group/average tumor volume in control group. Results were shown in FIG. 6, and it can be seen that the CAR NK-92 cell libraries targeting tumor tissues exhibited a very strong anti-tumor effect on each model.

### EXAMPLE 12

### Construction of an Individualized Natural Fully Human CAR-T Cell Library

This example was implemented with the natural human-derived phage scFV library constructed with PBMCs of 200 healthy volunteers in Example 8.

PBMCs of a lung cancer subject and a precancerous tissue of lung cancer obtained from surgery were taken as a control cell/tissue, and then three rounds of negative screening were conducted to remove phage capable of binding to the control cell/tissue in the phage natural human scFV library. A phage antibody library obtained after the screening was subjected to PCR amplification to obtain an antibody gene library. As tested, the library capacity did not change significantly.

The PBMCs of the above lung cancer subject were taken and then T lymphocytes were further isolated to further prepare a cell library, and CARs and genetic circuits were constructed according to the same method as in Example 1. The same genetic circuit and cell library construction methods as in Example 1 were adopted. Two controlled gene expression cassettes for the genetic circuits were shown in FIG. 2.

The obtained CAR cell library was named KRAB-iCasp9-CAR-hT, which had a library capacity of 6×10⁵.

### EXAMPLE 13

### Immunotherapy of Human Lung Cancer with the Individualized Natural Fully Human CAR-T Cell Library

The above KRAB-iCasp9-CAR-hT library was administered intravenously to the lung cancer subject in Example 12. The general administration route and dose for CAR-T cells (Fry T J, et al. Nature medicine, 2018, 24 (1): 20.) were adopted.

### EXAMPLE 14

### Treatment of IBD Small Animal Models with the Totally-Synthetic Mouse-Derived CAR-T Cell Library

This example was implemented with the KRAB-iCasp9-CAR-T cell library obtained in Example 1.

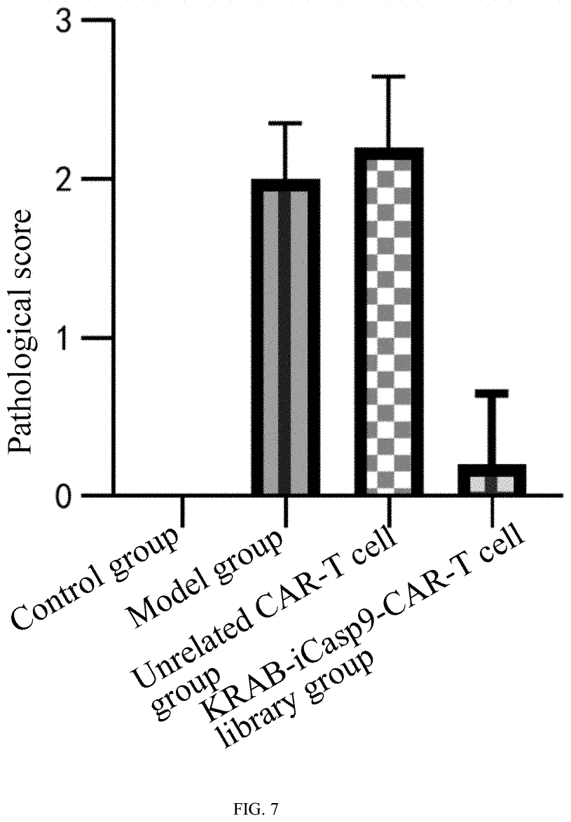

According to the method in the literature (Tian, Yuhua, et al. Gastroenterology 156.8 (2019): 2281-2296.), colitis was induced with trinitrobenzene sulfonic acid (TNBS) in BALB/c mice to obtain the IBD models. The model evaluation was conducted by the same method as in the literature (He C, et al. Gut, 2015.). The model mice were divided into a control group, a model group, an irrelevant CAR-T cell group, and a KRAB-iCasp9-CAR-T cell library group. The control group was not administered with TNBS, and the remaining groups were each administered with both low-dose TNBS and a therapeutic agent. The model group was administered with PBS; and the irrelevant CAR-T cell group was intravenously injected with CD19-CAR-T cells (the controlled gene expression cassette shown in FIG. 1C) at a dose of 5×10⁶ cells once every 2 d, 3 times in total. Four weeks later, case scoring was conducted by the same method as in the literature [Tian, Yuhua, et al. Gastroenterology 156.8 (2019): 2281-2296.]. Results were shown in FIG. 7, and it can be seen that the KRAB-iCasp9-CAR-T cell library exhibited a prominent therapeutic effect.

### EXAMPLE 15

### Treatment of Endometriosis Mouse Graft Models with the Natural Fully Human CAR NK-92 Cell Library

This example was implemented with the KRAB-iCasp9-CAR-NK92 library obtained in Example 8.

According to the model construction method in the literature (Masuda H, et al. N Proceedings of the National Academy of Sciences, 2007, 104 (6): 1925-1930.), an ectopic endometrium from an endometriosis subject was transplanted into NOG mice. After the mouse models were successfully constructed, the mice were divided into a control group, an irrelevant CAR-NK92 cell group (anti-CD19 CAR-NK92, including the controlled gene expression cassette shown in FIG. 2C), and a KRAB-iCasp9-CAR-NK92 library group. There were 10 mice in each group. The same therapeutic dose and manner as in Example 1 were adopted. At the second week of treatment, each group was administered with an iCasp9 inducer. Mice were sacrificed after 5 weeks of treatment to examine the transplanted endometrium. Results showed that, in the control group and irrelevant CAR-NK92 cell group, the endometrial tissue survived in each mouse; and in the KRAB-iCasp9-CAR-NK92 library treatment group, no endometrial tissue survived in mice.

### EXAMPLE 16

### Construction of a Natural Fully Human CAR-T Cell Library Targeting an Ectopic Endometrium

A natural human-derived phage scFV library was constructed according to the method in Example 8, and the method was briefly described as follows: a natural human-derived phage scFV library was constructed with PBMCs of 200 healthy volunteers. According to library capacity evaluation, the natural human-derived phage scFV library had a library capacity of 1×10¹⁰.

PBMCs of an endometriosis subject and an in situ endometrial tissue obtained from surgery were taken as a control cell/tissue, and then three rounds of negative screening were conducted to remove phage capable of binding to the control cell/tissue in the natural human-derived phage scFV library. A phage antibody library obtained after the screening was subjected to amplification, and then an antibody gene library was obtained through a general genetic engineering technology. As tested, the library capacity did not change significantly.

PBMCs of the above endometriosis subject were taken and then T lymphocytes were further isolated to further prepare a cell library, and CARs and genetic circuits were constructed according to the same method as in Example 1. The same genetic circuit construction as in Example 1 was adopted. Two controlled gene expression cassettes for the genetic circuits were shown in FIG. 2. The obtained natural fully human CAR-T cell library targeting an ectopic endometrium was named endo-KRAB-iCasp9-CAR-hT, which had a library capacity of 3×10⁵.

### EXAMPLE 17

### Treatment of Human Endometriosis with the Natural Fully Human CAR-T Cell Library Targeting an Ectopic Endometrium

The above endo-KRAB-iCasp9-CAR-hT library was administered intravenously to the endometriosis subject in Example 16. The general administration route and dose for CAR-T cells were adopted.

### EXAMPLE 18

### CAR NK-92 Cell Library including 495 Artificial Antibodies

A scFV library was first constructed through total synthesis. The antibody library was constructed from the following 495 mAbs: ABAGOVOMAB, ABCIXIMAB, ABELACIMAB, ABITUZUMAB, ABREZEKIMAB, ABRILUMAB, ACTOXUMAB, ADALIMUMAB, ADECATUMUMAB, ADUCANUMAB, AFASEVIKUMAB, AFELIMOMAB, ALACIZUMAB, ALEMTUZUMAB, ALIROCUMAB, AMATUXIMAB, ANATUMOMAB, ANDECALIXIMAB, ANETUMAB, ANIFROLUMAB, ANRUKINZUMAB, APRUTUMAB, ASCRINVACUMAB, ASELIZUMAB, ATIDORTOXUMAB, ATINUMAB, ATOLTIVIMAB, ATOROLIMUMAB, AVELUMAB, AZINTUXIZUMAB, BALSTILIMAB, BAPINEUZUMAB, BASILIXIMAB, BAVITUXIMAB, BECTUMOMAB, BEDINVETMAB, BEGELOMAB, BELANTAMAB, BELIMUMAB, BEMARITUZUMAB, BERLIMATOXUMAB, BERSANLIMAB, BERTILIMUMAB, BESILESOMAB, BEVACIZUMAB, BIMAGRUMAB, BIMEKIZUMAB, BIRTAMIMAB, BIVATUZUMAB, BLESELUMAB, BLINATUMOMAB, BLONTUVETMAB, BLOSOZUMAB, BOCOCIZUMAB, BRAZIKUMAB, BRIAKINUMAB, BROLUCIZUMAB, BRONTICTUZUMAB, BUDIGALIMAB, BUROSUMAB, CABIRALIZUMAB, CAMIDANLUMAB, CAMRELIZUMAB, CANAKINUMAB, CANTUZUMAB, CAPLACIZUMAB, CAPROMAB, CARLUMAB, CAROTUXIMAB, CATUMAXOMAB, CEDELIZUMAB, CEMIPLIMAB, CENDAKIMAB, CERGUTUZUMAB, CERTOLIZUMAB, CETRELIMAB, CETUXIMAB, CIBISATAMAB, CINPANEMAB, CITATUZUMAB, CIXUTUMUMAB, CLAZAKIZUMAB, CLENOLIXIMAB, CLIVATUZUMAB, COBOLIMAB, CODRITUZUMAB, COFETUZUMAB, COLTUXIMAB, CONATUMUMAB, CONCIZUMAB, COSFROVIXIMAB, CRENEZUMAB, CRIZANLIZUMAB, CROTEDUMAB, CROVALIMAB, CUSATUZUMAB, DACETUZUMAB, DACLIZUMAB, DALOTUZUMAB, DAPIROLIZUMAB, DECTREKUMAB, DEMCIZUMAB, DENINTUZUMAB, DENOSUMAB, DEPATUXIZUMAB, DETUMOMAB, DEZAMIZUMAB, DILPACIMAB, DINUTUXIMAB, DIRIDAVUMAB, DISITAMAB, DOMAGROZUMAB, DONANEMAB, DORLIMOMAB, DOSTARLIMAB, DROZITUMAB, DULIGOTUZUMAB, DUPILUMAB, DUSIGITUMAB, DUVORTUXIZUMAB, ECROMEXIMAB, EDOBACOMAB, EDRECOLOMAB, EFALIZUMAB, EFUNGUMAB, ELDELUMAB, ELEZANUMAB, ELGEMTUMAB, ELIPOVIMAB, ELSILIMOMAB, EMACTUZUMAB, EMIBETUZUMAB, EMICIZUMAB, ENAPOTAMAB, ENAVATUZUMAB, ENFORTUMAB, ENLIMOMAB, ENOBLITUZUMAB, ENOKIZUMAB, ENOTICUMAB, ENSITUXIMAB, ENVAFOLIMAB, EPITUMOMAB, EPTINEZUMAB, ERLIZUMAB, ERTUMAXOMAB, ETIGILIMAB, ETOKIMAB, ETROLIZUMAB, EVINACUMAB, EXBIVIRUMAB, FARALIMOMAB, FARICIMAB, FARLETUZUMAB, FASINUMAB, FELVIZUMAB, FEZAKINUMAB, FICLATUZUMAB, FIGITUMUMAB, FIRIVUMAB, FLANVOTUMAB, FLETIKUMAB, FLOTETUZUMAB, FONTOLIZUMAB, FORALUMAB, FORAVIRUMAB, FRESOLIMUMAB, FROVOCIMAB, FRUNEVETMAB, FULRANUMAB, FUTUXIMAB, GALIXIMAB, GANCOTAMAB, GANITUMAB, GANTENERUMAB, GARADACIMAB, GARETOSMAB, GAVILIMOMAB, GEDIVUMAB, GEMTUZUMAB, GEVOKIZUMAB, GILVETMAB, GIMSILUMAB, GIRENTUXIMAB, GLEMBATUMUMAB, GLENZOCIMAB, GOLIMUMAB, GOSURANEMAB, IANALUMAB, IBRITUMOMAB, ICRUCUMAB, IDARUCIZUMAB, IERAMILIMAB, IFABOTUZUMAB, IGOVOMAB, ILADATUZUMAB, IMALUMAB, IMAPRELIMAB, IMCIROMAB, IMGATUZUMAB, INCLACUMAB, INDATUXIMAB, INDUSATUMAB, INEBILIZUMAB, INFLIXIMAB, INOLIMOMAB, INOTUZUMAB, INTETUMUMAB, APAMISTAMAB, DERLOTUXIMAB, IPILIMUMAB, IRATUMUMAB, ISATUXIMAB, ISCALIMAB, ISTIRATUMAB, IXEKIZUMAB, KELIXIMAB, LABETUZUMAB, LACNOTUZUMAB, LACUTAMAB, LADIRATUZUMAB, LAMPALIZUMAB, LANADELUMAB, LANDOGROZUMAB, LAPRITUXIMAB, LARCAVIXIMAB, LEBRIKIZUMAB, LEMALESOMAB, LENVERVIMAB, LENZILUMAB, LERDELIMUMAB, LERONLIMAB, LESOFAVUMAB, LETOLIZUMAB, LEVILIMAB, LEXATUMUMAB, LIBIVIRUMAB, LIFASTUZUMAB, LIGELIZUMAB, LILOTOMAB, LINTUZUMAB, LIRILUMAB, LODELCIZUMAB, LONCASTUXIMAB, LORVOTUZUMAB, LOSATUXIZUMAB, LUCATUMUMAB, LULIZUMAB, LUMILIXIMAB, LUMRETUZUMAB, LUPARTUMAB, LUTIKIZUMAB, MAFTIVIMAB, MAGROLIMAB, MAPATUMUMAB, MARGETUXIMAB, MARSTACIMAB, MASLIMOMAB, MATUZUMAB, MAVRILIMUMAB, MEPOLIZUMAB, METELIMUMAB, MILATUZUMAB, MINRETUMOMAB, MIRIKIZUMAB, MIRVETUXIMAB, MITAZALIMAB, MITUMOMAB, MODOTUXIMAB, MOGAMULIZUMAB, MONALIZUMAB, MOROLIMUMAB, MOSUNETUZUMAB, MOTAVIZUMAB, MURLENTAMAB, NACOLOMAB, NAMILUMAB, NAPTUMOMAB, NARATUXIMAB, NARNATUMAB, NATALIZUMAB, NAVICIXIZUMAB, NAVIVUMAB, NAXITAMAB, NEBACUMAB, NEMOLIZUMAB, NERELIMOMAB, NESVACUMAB, NETAKIMAB, NIDANILIMAB, NIMACIMAB, NIMOTUZUMAB, NIRSEVIMAB, NIVOLUMAB, OBEXELIMAB, OBILTOXAXIMAB, OBINUTUZUMAB, OCARATUZUMAB, ODULIMOMAB, OFATUMUMAB, OLECLUMAB, OLENDALIZUMAB, OLINVACIMAB, OLOKIZUMAB, OMALIZUMAB, OMBURTAMAB, ONARTUZUMAB, ONTAMALIMAB, ONTUXIZUMAB, ONVATILIMAB, OPICINUMAB, OREGOVOMAB, ORILANOLIMAB, ORTICUMAB, OSOCIMAB, OTELIXIZUMAB, OTILIMAB, OTLERTUZUMAB, OXELUMAB, OZANEZUMAB, OZORALIZUMAB, PAGIBAXIMAB, PALIVIZUMAB, PAMREVLUMAB, PANITUMUMAB, PANOBACUMAB, PARSATUZUMAB, PASCOLIZUMAB, PASOTUXIZUMAB, PATECLIZUMAB, PATRITUMAB, PEMBROLIZUMAB, PEPINEMAB, PERAKIZUMAB, PERTUZUMAB, PEXELIZUMAB, PIDILIZUMAB, PINATUZUMAB, PLACULUMAB, PLAMOTAMAB, PLOZALIZUMAB, POLATUZUMAB, PONEZUMAB, PORGAVIXIMAB, POZELIMAB, PRASINEZUMAB, PREZALUMAB, PRILIXIMAB, PRITOXAXIMAB, PRITUMUMAB, PROLGOLIMAB, QUETMOLIMAB, QUILIZUMAB, RACOTUMOMAB, RADRETUMAB, RAFIVIRUMAB, RALPANCIZUMAB, RANEVETMAB, RANIBIZUMAB, RAVAGALIMAB, RAXIBACUMAB, REFANEZUMAB, REGAVIRUMAB, RELATLIMAB, RELFOVETMAB, REMTOLUMAB, RESLIZUMAB, RILOTUMUMAB, RINUCUMAB, RITUXIMAB, RIVABAZUMAB, ROBATUMUMAB, ROLINSATAMAB, ROMILKIMAB, RONTALIZUMAB, ROSMANTUZUMAB, ROVALPITUZUMAB, ROZANOLIXIZUMAB, SACITUZUMAB, SAMALIZUMAB, SAMROTAMAB, SARILUMAB, SATRALIZUMAB, SATUMOMAB, SECUKINUMAB, SELICRELUMAB, SEMORINEMAB, SERCLUTAMAB, SERIBANTUMAB, SETOXAXIMAB, SETRUSUMAB, SIBROTUZUMAB, SIFALIMUMAB, SIMTUZUMAB, SINTILIMAB, SIRTRATUMAB, SIRUKUMAB, SOFITUZUMAB, SOLANEZUMAB, SOLITOMAB, SONTUZUMAB, SPARTALIZUMAB, SPESOLIMAB, STAMULUMAB, SULESOMAB, SUPTAVUMAB, SUTIMLIMAB, SUVIZUMAB, SUVRATOXUMAB, TABALUMAB, TABITUXIMAB, TADOCIZUMAB, TAFASITAMAB, TALACOTUZUMAB, TALIZUMAB, TAMRINTAMAB, TAMTUVETMAB, TANEZUMAB, TAPLITUMOMAB, TAREXTUMAB, TAVOLIMAB, FANOLESOMAB, NOFETUMOMAB, PINTUMOMAB, TECLISTAMAB, TEFIBAZUMAB, TELIMOMAB, TELISOTUZUMAB, TEMELIMAB, TENATUMOMAB, TENELIXIMAB, TEPLIZUMAB, TEPODITAMAB, TEPROTUMUMAB, TESIDOLUMAB, TEZEPELUMAB, TIBULIZUMAB, TIDUTAMAB, TIGATUZUMAB, TILAVONEMAB, TILDRAKIZUMAB, TIMOLUMAB, TIRAGOLUMAB, TISLELIZUMAB, TISOTUMAB, TOCILIZUMAB, TOMARALIMAB, TORALIZUMAB, TORIPALIMAB, TOSATOXUMAB, TOSITUMOMAB, TOVETUMAB, TRALOKINUMAB, TRASTUZUMAB, TREGALIZUMAB, TREMELIMUMAB, TREVOGRUMAB, TUCOTUZUMAB, TUVIRUMAB, UBLITUXIMAB, ULOCUPLUMAB, URELUMAB, URTOXAZUMAB, USTEKINUMAB, UTOMILUMAB, VADASTUXIMAB, VANDORTUZUMAB, VANTICTUMAB, VANUCIZUMAB, VAPALIXIMAB, VARISACUMAB, VARLILUMAB, VATELIZUMAB, VELTUZUMAB, VEPALIMOMAB, VESENCUMAB, VIBECOTAMAB, VISILIZUMAB, VOBARILIZUMAB, VOFATAMAB, VOLAGIDEMAB, VOLOCIXIMAB, VONLEROLIZUMAB, VOPRATELIMAB, VORSETUZUMAB, VOTUMUMAB, VUNAKIZUMAB, XENTUZUMAB, ZALIFRELIMAB, ZAMPILIMAB, ZANOLIMUMAB, ZENOCUTUZUMAB, ZIRALIMUMAB, ZOLBETUXIMAB, and ZOLIMOMAB.

NK-92 cells were adopted to further construct a cell library. CARs and intracellular genetic circuits were constructed according to the same method as in Example 1 to obtain the CAR NK-92 cell library including 495 artificial antibodies. The obtained CAR NK-92 cell library was named 495-KRAB-iCasp9-CAR-NK92, which had a library capacity of 495.

### EXAMPLE 19

### Immunotherapy of Human Pancreatic Cancer with the CAR NK-92 Cell Library including 495 Artificial Antibodies

The above 495-KRAB-iCasp9-CAR-NK92 library was administered intravenously to a pancreatic cancer subject. The general administration dose, frequency, and route for cell therapy were adopted.

### EXAMPLE 20

### Example Overview of Vector Assembly Construction Methods

(1) Construction Samples of a First Genetic Element

The first genetic element is a CAR library. CAR is an artificial receptor, and the construction of the CAR library can be found in the patent document WO2015/123642. The present disclosure emphasizes the use of a randomized library of extracellular recognition domains of CARs to realize the applications of specific CARs. Examples of construction methods are as follows.

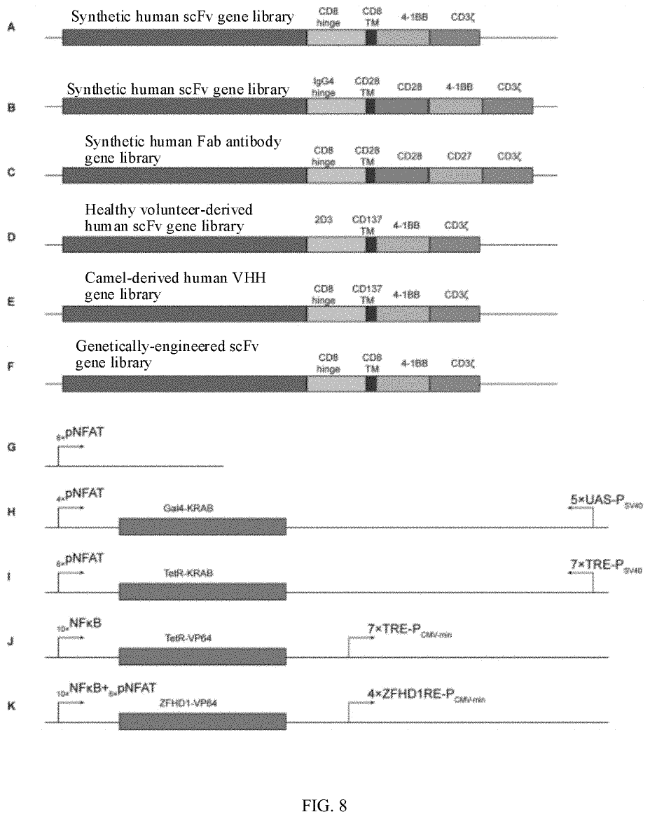

Construction as follows: A human antibody gene library is artificially constructed through DNA synthesis according to antibody coding rules of human genes. According to a CAR scheme, a human scFV gene library CD8 hinge-CD8TM-4-1BB-CD3ζ shown in FIG. 8A is constructed.

Construction as follows: A human antibody gene library is artificially constructed through DNA synthesis according to antibody coding rules of human genes. The gene library is constructed into a phage vector, and then the phage is amplified to obtain a phage antibody library. If the screening is to be conducted in an animal model, the phage antibody library is administered to the animal model, and the phage capable of binding to an antigen of the animal model is subtracted to obtain a phage antibody sub-library; and then an antibody gene library of the sub-library is obtained by a genetic engineering method. According to a CAR scheme, a scFV library IgG4-hinge-CD28TM-CD28-4-1BB-CD3ζ shown in FIG. 8B is constructed. If it is to be directly used in a subject, a control tissue of the subject (such as PBMCs and a paracancerous tissue of the subject) is collected, and the phage capable of binding to the control tissue is subtracted to obtain a phage antibody sub-library; and then an antibody gene library of the sub-library is obtained by a genetic engineering method. According to a CAR scheme, a Fab antibody library CD8-hinge-CD28TM-CD28-CD27-CD3ζ shown in FIG. 8C is constructed.

Construction as follows: An antibody gene library is constructed by a genetic engineering method with PBMCs of multiple healthy volunteers, then the gene library is constructed into a yeast vector, and the yeast is amplified to obtain a yeast antibody library. According to a CAR scheme, a human antibody gene library 2D3-CD137TM-4-1BB-CD3ζ shown in FIG. 8D is constructed.

Construction as follows: A camel antibody gene library is constructed by a genetic engineering method with PBMCs of a camel, and according to a CAR scheme, a camel antibody gene library CD8-hinge-CD8TM-4-1BB-CD3ζ shown in FIG. 8E is constructed.

Construction as follows: An antibody gene library is constructed by a genetic engineering method with PBMCs of multiple healthy volunteers, then the gene library is constructed into a yeast vector, and the yeast is amplified to obtain a yeast antibody library. The tumor cell line MDA-MB-231 is allowed to contact the antibody library, and yeast cells capable of binding to the tumor cells are screened out. A sub-library of the antibody gene library in the yeast cells is obtained by a genetic engineering method, then an antibody library is re-constructed by genetic engineering methods of CDR mutagenesis, affinity maturation, and strand exchange, and according to a CAR scheme, a genetically-engineered scFV library CD8 hinge-CD8TM-4-1BB-CD3ζ shown in FIG. 8F is constructed.

(2) Construction Samples of a Second Genetic Element

The second genetic element is a genetic circuit. Construction as follows: 6 NFAT-responsive elements and a minimal IL-2 promoter are fused (6×NFAT), as shown in FIG. 8G.

Construction as follows: 4 NFAT-responsive elements and a minimal IL-2 promoter are fused (4×NFAT), and Gal4-KRAB is included downstream of a resulting fusion; and then a 5×UAS-P_{SV40} fusion promoter regulated by Gal4-KRAB is constructed, as shown in FIG. 8H.

Construction as follows: 6 NFAT-responsive elements and a minimal IL-2 promoter are fused (6×NFAT), and the transcription factor TetR-KRAB is included downstream of a resulting fusion; and then a 7×TRE-P_{SV40} fusion promoter regulated by TetR-KRAB is constructed, as shown in FIG. 8I.

Construction as follows: 10 NFκB-binding elements and a minimal HIVtata promoter are fused (10×NFκB), and the transcription factor TetR-VP64 is included downstream of a resulting fusion; and then 7 TREs and a minimal CMV promoter are fused to obtain a fusion promoter regulated by TetR-VP64 (7×TRE-P_{CMV-min}), as shown in FIG. 8J.

Construction as follows: 10 NFκB-binding elements, 6 NFAT-responsive elements, and a minimal IL-2 promoter are fused (10×NFκB+6×NFAT), and the transcription factor ZFHD1-VP64 is included downstream of a resulting fusion; and then a 4×ZFHD1RE-P_{CMV-min} promoter regulated by ZFHD1-VP64 is constructed, as shown in FIG. 8K.

(3) Construction Samples of a Third Genetic Element

The third genetic element is a genetic element encoding a protein, and the protein may include a puromycin resistance protein (PuroR), a neomycin resistance protein (NeoR), a blasticidin resistance protein (Blasticidin-R), a hygromycin B resistance protein (Hygromycin B-R), an HSV-TK protein, a CD protein, or an iCasp9 suicide system protein.

(4) Examples of Vector Assembly

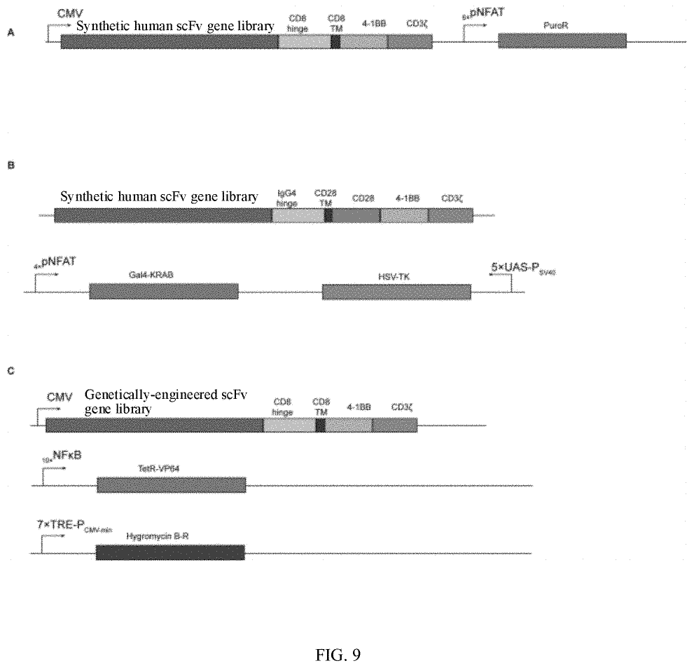

A vector assembly including three genetic elements can be constructed as follows: (i) A human antibody gene library is artificially constructed according to antibody coding rules of human genes. According to a CAR scheme, a human scFV gene library CD8 hinge-CD8TM-4-1BB-CD3 is constructed, that is, a first genetic element shown in FIG. 2A is constructed. (ii) 6 NFAT-responsive elements and a minimal IL-2 promoter (6×NFAT) are fused to construct a second genetic element shown in FIG. 2G. (iii) A third genetic element encoding a puromycin resistance protein (PuroR) is constructed. These elements are inserted into a vector shown in FIG. 9A as a whole, and the vector may include another regulatory element described in the prior art, such as a CMV promoter.

The vector assembly can also be constructed as follows: (i) A human antibody gene library is artificially constructed through DNA synthesis according to antibody coding rules of human genes. The gene library is constructed into a phage vector, and then the phage is amplified to obtain a phage antibody library. If the screening is to be conducted in an animal model, the phage antibody library is administered to the animal model, and the phage capable of binding to an antigen of the animal model is subtracted to obtain a phage antibody sub-library; and then an antibody gene library of the sub-library is obtained by a genetic engineering method. According to a CAR scheme, a scFV library IgG4-hinge-CD28TM-CD28-4-1BB-CD3ζ is constructed, that is, a first genetic element shown in FIG. 2B is constructed. (ii) 4 NFAT-responsive elements and a minimal IL-2 promoter (4×NFAT) are fused to obtain a fusion promoter with Gal4-KRAB downstream, then a 5×UAS-P_{SV40} fusion promoter regulated by Gal4-KRAB is constructed, and then a second genetic element shown in FIG. 2H is constructed with the fusion promoters. (iii) A third genetic element encoding an HSV-TK protein is constructed. These elements are inserted into two vectors shown in FIG. 9B as a whole.

The vector assembly can also be constructed as follows: (i) An antibody gene library is constructed by a genetic engineering method with PBMCs of multiple healthy volunteers, then the gene library is constructed into a yeast vector, and the yeast is amplified to obtain a yeast antibody library. The tumor cell line MDA-MB-231 is allowed to contact the antibody library, and yeast cells capable of binding to the tumor cells are screened out. A sub-library of the antibody gene library in the yeast cells is obtained by a genetic engineering method, then an antibody library is re-constructed by genetic engineering methods of CDR mutagenesis, affinity maturation, and strand exchange, and according to a CAR scheme, a genetically-engineered scFV library CD8 hinge-CD8TM-4-1BB-CD3ζ is constructed, that is, a first genetic element shown in FIG. 2F is constructed. (ii) 10 NFκB-binding elements and a minimal HIVtata promoter are fused to obtain a fusion promoter (10×NFκB) with the transcription factor TetR-VP64 downstream, then 7 TREs and a minimal CMV promoter are fused to obtain a fusion promoter regulated by TetR-VP64 (7×TRE-P_{CMV-min}), and then a second genetic element shown in FIG. 2J is constructed with the fusion promoters. (iii) A third genetic element encoding a hygromycin B resistance protein (Hygromycin B-R) is constructed. These elements are inserted into three vectors shown in FIG. 9C as a whole.

### EXAMPLE 21

### Treatment of a Subject Exposed to an Unknown Pathogen

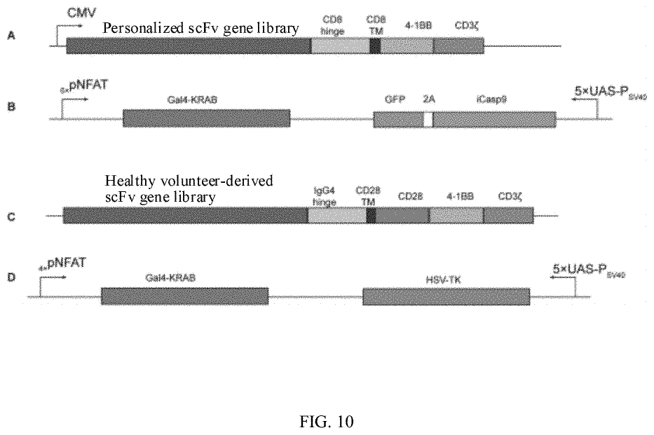

An individualized CAR cell library was first constructed. An antibody gene library was constructed by a genetic engineering method with PBMCs of healthy volunteers, and according to a CAR scheme, an individualized scFV library CD8 hinge-CD8TM-4-1BB-CD3ζ was constructed, as shown in FIG. 10A. A second genetic element including 6xNFAT with the transcription factor Gal4-KRAB downstream and 5×UAS-P_{SV40} regulated by Gal4-KRAB was constructed. A third genetic element iCasp9 was constructed, and a vector was constructed according to the manner in FIG. 10B. Other regulatory elements described in the prior art on the vector, such as a CMV promoter, a 2A cleaving peptide, and a green fluorescent protein, can also be seen in FIG. 10A and FIG. 10B. The above vector was transfected into T cells of a subject to obtain the CAR cell library, and the CAR cell library was cryopreserved.

In a public safety emergency or bioterrorism attack, people are exposed to unknown pathogens or toxins. The exposure is in many different modes, such as food or water intake, aerosol inhalation, or skin contact. The pathogens may include Bacillus anthracis (anthrax), influenza virus, smallpox virus, Yersinia pestis (pestis), Ebola or Marburg virus, Francisella tularensis (tularemia), Hantavirus, dengue virus, cholera toxin, botulinum toxin, ricin toxin, Salmonella, Escherichia coli (E. coli) such as E. coli 0157:H7, Shigella, and Listeria, for example. When a threatening microbe has not been identified, some patients have developed severe illness with similar symptoms, including high fever, chills, cough, severe fatigue, and diarrhea. Patients can receive a standard therapy, such as administration of an antiviral drug, an antibiotic, an antitoxin, and an immunoglobulin. The CAR cell library and the composition of the present disclosure can be intravenously administered to a patient with infection symptoms and inflammation signs (such as fever and chills) as a preventive measure or a treatment means for the patient, such as a composition with 3×10⁹ CAR cells as an active component. Once distributed into a body fluid (especially blood), the cell library can clear lesion cell and inflammatory mediators. By removing these disease-associated mediators, the cell library reduces the triggers of additional systemic inflammation in the patient and reduces the generation of systemic inflammatory mediators such as cytokines, thereby preventing or limiting the cell death, organ damage, multiple-organ failure (MOF), and potential death induced by cytokines or other inflammatory mediators. The cell library may be administered once or repeatedly over a period of hours to days to achieve a persistent or stable effect.

At an appropriate time, an inducer was administered to a patient, then CAR-inactivated library cells were removed, and a cell library was enriched from the patient. An extracellular recognition domain of CAR was obtained by a genetic engineering method, and accordingly, an antibody, a CAR, and an engineered cell were further prepared and used in the treatment of other patients.

### EXAMPLE 22

### Administration of a CAR Cell Library to an IBD Patient

A healthy human-derived CAR cell library was first constructed. An antibody gene library was constructed by a genetic engineering method with PBMCs of at least 100 healthy volunteers. Then a phage antibody library was constructed, and with PBMCs of the patient as a control tissue, the background was subtracted. Then an antibody gene library was obtained by an antibody engineering method. According to a CAR scheme, a healthy volunteer-derived scFV gene library IgG4 hinge-CD28TM-CD28-4-1BB-CD3ζ was constructed, as shown in FIG. 10C. A second genetic element including 4×NFAT with the transcription factor Gal4-KRAB downstream and 5×UAS-P_{SV40} regulated by Gal4-KRAB was constructed. A third genetic element HSV-TK was constructed, and a vector was constructed according to the manner in FIG. 10D. Other regulatory elements described in the prior art on the vector can also be seen in FIG. 10C and FIG. 10D. The above vector was transfected into T cells of the patient to obtain the CAR cell library, and the CAR cell library was cryopreserved.

When the IBD patient worsened to persistent severe diarrhea, the patient was administered with a standard therapy, including systemic corticosteroid and parenteral TNF blocker therapies. As a part of anti-inflammation therapy for the patient, an effective dose of the cell library was administered to the patient, such as a composition with 3×10⁹ CAR cells as an active component. The cell library cleared abnormal cells and neutralized inflammatory mediators generated locally (including cytokines generated in an intestinal tract), which promoted the healing of bowels and prevented the recurrence.

### EXAMPLE 23

### Administration of a Cell Library to a Burn Patient

A burn patient suffering from severe burns of more than 30% of his total body surface area (TBSA) and lung damage caused by prolonged smoke and chemical inhalation was selected. Although the debridement was conducted, the patient still developed severe SIRS and ARDS, requiring mechanical ventilation. The patient was administered with a standard therapy, mainly including a supportive care measure.

T cells of the patient were collected, the background was subtracted according to the method in Example 22, and then a CAR cell library was constructed. In response to systemic inflammation capable of rapidly causing MOF, the patient was administered with a composition including 3×10⁹ CAR cells as an active component to inhibit the systemic inflammation. The cell library can remove systemic inflammatory mediators that can result in the production of more inflammatory mediators.

### EXAMPLE 24

### Administration of a Cell Library to an Endometritis Patient

An endometritis patient with infertility was selected, whose conditions were not improved after the treatment with antibiotics, hormones, and traditional Chinese medicines (TCMs).

T cells of the patient were collected, the patient background was subtracted according to the method in Example 22, and then a CAR cell library was constructed. In response to endometritis, the patient was administered with a composition including 3×10⁹ CAR cells as an active component to inhibit the endometritis. After a therapeutic effect was achieved, the patient was administered with an inducer to remove the cell library.

### EXAMPLE 25

### Administration of a Cell Library to an Anti-Aging Subject

A subject who wished to remove aging cells and achieve an anti-aging effect was selected.

T cells of the subject were collected, the subject background was subtracted according to the method in Example 21, and then a CAR cell library was constructed.

In response to anti-aging, the patient was administered with a composition including 3×10⁷ CAR cells as an active component to achieve the anti-aging effect. Skin, biochemical indexes, and the like were detected after the treatment. After a therapeutic effect was achieved, the patient was administered with an inducer to remove the cell library. One month later, the skin elasticity of the patient was enhanced, and the liver function of the patient was significantly improved.

### EXAMPLE 26

### Administration of a Cell Library to an Alzheimer's Disease Patient

A high-risk Alzheimer's disease potential patient in a healthy state was selected, and an individualized CAR cell library was first constructed. According to a CAR scheme, an individualized scFV library CD8 hinge-CD8TM-4-1BB-CD3ζ was constructed, as shown in FIG. 10A. A second genetic element including 6×NFAT with the transcription factor Gal4-KRAB downstream and 5×UAS-P_{SV40} regulated by Gal4-KRAB was constructed. A third genetic element iCasp9 was constructed, and a vector was constructed according to the manner in FIG. 10B. Other regulatory elements described in the prior art on the vector, such as a CMV promoter, a 2A cleaving peptide, and a green fluorescent protein, can also be seen in FIG. 10A and FIG. 10B. The above vector, a Sleeping Beauty (SB) transposon vector, was transfected into T cells of a subject by an electrochemical method to obtain the CAR cell library, and the CAR cell library was cryopreserved.

After a period of time, the patient developed Alzheimer's disease, which could not be alleviated by existing supportive treatments and continued to deteriorate. An effective dose of the cell library was intradurally injected into the patient, such as a composition with 1×10⁵ CAR cells as an active component. The cell library cleared abnormal neurons and reduced neurological symptoms. After a therapeutic effect was achieved, the patient was administered with an inducer to remove the cell library.

### EXAMPLE 27

### Administration of a Cell Library to an Infertility Patient

A patient diagnosed with immune infertility that could not be cured with the existing methods was selected. T cells of the patient were collected, the patient background was subtracted according to the method in Example 21, and then a CAR cell library was constructed. In response to abnormal immune factors in the utero, the patient was administered with a composition including 3×10⁷ CAR cells as an active component to remove abnormal immune mediators. At an appropriate time, the patient was administered with an inducer to remove the cell library. The patient eventually became pregnant.

### EXAMPLE 28

### Administration of a Cell Library to a Liver Fibrosis Patient

A patient who had suffered from hepatitis B 10 years ago and was diagnosed with partial liver fibrosis was selected, and the disease failed to be cured with multiple anti-fibrosis treatments and continued to progress. T cells of the patient were collected, the patient background was subtracted according to the method in Example 22, and then a CAR cell library was constructed. In response to abnormal immune factors in the liver, the patient was administered with a composition including 3×10⁷ CAR cells as an active component to remove abnormal intrahepatic immune mediators and diseased cells. After a therapeutic effect was achieved, the patient was administered with an inducer to remove the cell library. The degree of fibrosis in the patient was successfully delayed.

### EXAMPLE 29

### Administration of a Cell Library to a Chronic Pelvic Inflammatory Disease (CPID) Patient

A CPID patient with infertility was selected, whose conditions were not improved after the treatment with antibiotics, hormones, and TCMs.

T cells of the patient were collected, the patient background was subtracted according to the method in Example 22, and then a CAR cell library was constructed. In response to CPID, the patient was administered with a composition including 3×10⁹ CAR cells as an active component to inhibit the CPID. After a therapeutic effect was achieved, the patient was administered with an inducer to remove the cell library.

### EXAMPLE 30

### Cosmetic Repair of Multiple Skin Scars

A patient with multiple skin scars was selected, and the surgery, laser, and other treatments led to an insignificant therapeutic effect for the skin scars.

T cells of the patient were collected, the patient background was subtracted according to the method in Example 22, and then a CAR cell library was constructed. In response to the multiple skin scars of the patient, the patient was administered with a composition including 1×10⁶ CAR cells as an active component to treat the multiple skin scars. After a therapeutic effect was achieved, the patient was administered with an inducer to remove the cell library.

### EXAMPLE 31

### Treatment of Lumbar Hyperosteogeny

A healthy patient was selected, and an antibody gene library of his PBMCs was preventively deposited, with a library capacity of 1×10⁶. A few years later, the patient developed severe lumbar hyperosteogeny, which could not be treated with a variety of existing treatments. T cells of the patient were collected, and then a CAR cell library was constructed according to the construction method in Example 21 and the gene library of the patient deposited. The patient was administered with a composition including 1×10⁶ CAR cells as an active component to treat the lumbar hyperosteogeny. After a therapeutic effect was achieved, the patient was administered with an inducer to remove the cell library.

### EXAMPLE 32

### Construction of a Common Target Library

A library was constructed according to a CAR scheme, and a scFv library of the CAR library was a small-scale library including the binding targets of CD19, BCMA, Mesothelin, GD2, EGFR, HER2, CD22, CD123, Glypican 3, CD30, MUC1, CD33, CD20, CD38, EpCAM, CD56, CD138, CD7, CD133, CEA, CD34, CD117, Claudin18.2, PSCA, cMET, Lewis Y, EphA2, NKG2D ligands, ErbB, NY-ESO-1, CLL-1, CD10, LI13Rα2, CD171, ROR2, AXL, Kappa, CS1, FAP, IL-1RAP, MG7, PSMA, CD5, ROR1, CD70, HER3, Gp75, phosphatidylserine, cMyc, CD4, CD44v6, CD45, CD28, CD3, CD3e, CD52, CD74, CD30, CD166, CD24, EGFR/HER3 fusions, carbohydrates, Aspergillus, Dectin, Ebolavirus, fungi, GP, HERV-K, VEGF-R2, TGF-2R, IgG4, biotin, O-AcGD2, Cadherin 2, OB-cadherin, α5β1 integrin, αVβ6 integrin, Syndecan-1, Cadherin 1, Claudin 12, Claudin 7, Claudin 3, and ZO-1. A small-scale scFv library IgG4 hinge-CD28TM-CD28-4-1BB-CD3ζ was constructed. A second genetic element including 4×NFAT with the transcription factor Gal4-KRAB downstream and 5×UAS-P_{SV40} regulated by Gal4-KRAB was constructed. A third genetic element HSV-TK was constructed. The above vector was transfected into NK-92 cells to obtain a CAR cell library, and the CAR cell library was cryopreserved.

### EXAMPLE 33

### Examples of In Vitro Screening

A variety of methods can be used to construct a CAR cell library. For example, a healthy human-derived CAR cell library is first constructed; an antibody gene library is constructed by a genetic engineering method with PBMCs of at least 100 healthy volunteers, and according to a CAR scheme, a healthy volunteer-derived scFv library IgG4 hinge-CD28TM-CD28-4-1BB-CD3ζ is constructed, as shown in FIG. 10C; a second genetic element including 4×NFAT with the transcription factor Gal4-KRAB downstream and 5×UAS-P_{SV40} regulated by Gal4-KRAB is constructed; a third genetic element HSV-TK is constructed, and a vector is constructed according to the manner in FIG. 10D, where other regulatory elements described in the prior art on the vector can also be seen in FIG. 10C and FIG. 10D; and the above vector is transfected into Jurkat cells to obtain a CAR cell library.

A target antigen is coated on a well plate, and then 1×10⁹ of Jurkat cells are added to each well; and at an appropriate time, an inducer is added to remove cells that do not bind to the antigen, the remaining jurkat cells are separated, and a CAR gene is obtained by a genetic engineering method for analysis.

The unexplained parts involved in the present disclosure are the same as the prior art or implemented by the prior art. The applicants declare that the detailed methods of the present disclosure are illustrated through the above examples, but the present disclosure is not limited to the above detailed methods, that is, it does not mean that the present disclosure must be implemented relying on the above detailed methods. Those skilled in the art should understand that any improvement to the present disclosure, equivalent replacement of each raw material of the product of the present disclosure, addition of auxiliary ingredients, selection of specific methods, and the like all fall within the protection scope and disclosure scope of the present disclosure.

What is claimed is:

1. A vector assembly, comprising one or more vectors and three genetic elements inserted into the one or more vectors, wherein the three genetic elements correspond to a plurality of first genetic elements encoding one or more idiotype chimeric antigen receptors (CARs), a second genetic element carrying one or more genetic circuits, and a third genetic element encoding one or more inducible proteins, respectively;
the one or more idiotype CARs each comprise an intracellular signaling domain, a transmembrane domain, and an extracellular recognition domain, and the extracellular recognition domain comprises an intact antibody, a heavy chain or light chain constituting an antibody, or an antibody fragment; when there are more than one idiotype CARs, at least three idiotype CARs are comprised;
the one or more genetic circuits are pre-programmed and each comprise one or more cis-regulatory factors and/or one or more transcription factors; the activation of the one or more idiotype CARs encoded by the plurality of first genetic elements leads to an expression regulation effect on the one or more inducible proteins encoded by the third genetic element; and
the one or more inducible proteins comprise one or two selected from the group consisting of a drug resistance protein and a suicide protein.

2. The vector assembly according to claim 1, wherein
the antibody fragment comprises an antibody variable region, a single-chain fragment variable (scFV), a single-domain antibody, or an antigen-binding fragment (Fab);
the one or more cis-regulatory factors comprise a single cis-acting factor and a fusion cis-acting factor, and the fusion cis-acting factor comprises a combination of one or more single cis-acting factors;
the single cis-acting factor comprises any one or a combination of at least two selected from the group consisting of an NFAT-responsive promoter element, an NFκB-responsive promoter element, a tetracycline responsive element, an upstream activating sequence (UAS) of a galactose-metabolizing enzyme gene promoter, a PIP responsive element, a ZFHD1 responsive element, a ZF21-16 responsive element, a ZF42-10 responsive element, a ZF43-8 responsive element, a ZF54-8 responsive element, a minimal CMV promoter, a CMV promoter, an SV40 promoter, a minimal IL-2 promoter, a minimal insect heat shock protein (HSP) 70 promoter, and a minimal HIVtata promoter;
the fusion cis-acting factor comprises any one or a combination of at least two selected from the group consisting of 4×NFAT, 6×NFAT, 5×NFκB, 10×NFκB, 7×TRE-P_{CMV-min}, 5×UAS-P_{CMV-min}, 4×PIR-P_{CMV-min}, 8×PIR-P_{CMV-min}, 8×PIR-Pₕₛₚ₇₀ₘᵢₙ, 4×ZFHD1RE-P_{CMV-min}, 8×ZF21-16RE-P_{CMV-min}, 8×ZF42-10RE-P_{CMV-min}, 8×ZZF43-8R-_{CMV-min}, 8×ZF54-8RE-P_{CMV-min}, 7×TRE-P_{SV40}, 7×TRE-Pcmv, 5×uAS-P_{SV40}, 4×PIR-P_{SV40}, 8×PIR-P_{SV40}, 4×ZFHD1RE-P_{SV40}, 8×ZF21-16RE-P_{SV40}, 8×ZF42-10RE-P_{SV40}, 8×ZF43-8RE-P_{SV40}, and 8×ZF54-8RE-P_{SV40};
the one or more transcription factors comprise any one or a combination of at least two selected from the group consisting of TetR-VP64 (tTA), Gal4-VP64, PIP-VP64, ZF21-16-VP64, ZF-42-10-VP64, ZF43-8-VP64, ZF54-8-VP64, ZFHD1-VP64, Gal4-KRAB, TetR-KRAB, PIP-KRAB, ZF21-16-KRAB, ZF-42-10-KRAB, ZF43-8-KRAB, ZF54-8-KRAB, and ZFHD1-KRAB;
the expression regulation effect comprises any one or a combination of at least two selected from the group consisting of activating transcription and expression, enhancing transcription and expression, terminating transcription and expression, and inhibiting transcription and expression;
the drug resistance protein comprises any one or a combination of at least two selected from the group consisting of a puromycin resistance protein, a neomycin resistance protein, a blasticidin resistance protein, and a hygromycin B resistance protein; and
the suicide protein comprises any one or a combination of at least two selected from the group consisting of a herpes simplex virus (HSV) thymidine kinase (TK) protein, a cytosine deaminase (CD) protein, and an inducible caspase 9 (iCasp9) suicide system protein.

3. The vector assembly according to claim 2, wherein
the one or more genetic circuits each are composed of (i) any one selected from the group consisting of a combination of Gal4-KRAB and 5×UAS-P_{SV40}, a combination of TetR-KRAB and 7×TRE-P_{SV40}, a combination of Gal4-VP64 and 5×UAS-P_{CMV-min}, a combination of TetR-VP64 and 7×TRE-P_{CMV-min}, and a combination of TetR-KRAB and 7×TRE-P_{cmv} and (ii) any one selected from the group consisting of 4×NFAT, 6×NFAT, 5×NFκB, and 10×NFκB.

4. A CAR cell library carrying the vector assembly according to claim 1.

5. A preparation and screening method of the CAR cell library according to claim 4, comprising:
inserting the plurality of first genetic elements, the second genetic element, and the third genetic element into the one or more vectors, transfecting the one or more vectors into cells to obtain the CAR cell library, and screening,
wherein a method for the transfecting comprises any one or a combination of at least two selected from the group consisting of viral transfection, chemical transfection, and electroporation transfection; and the cells are mammalian immune cells or genetically-engineered immune cells.

6. The preparation and screening method according to claim 5, comprising the following steps:
A. preparation of an antibody gene library
establishing the antibody gene library through a healthy volunteer source, a total synthesis process, and/or a genetic engineering process;
B. construction of genetic elements
constructing a first genetic element comprising the antibody gene library-CAR, wherein the antibody gene library or an antibody gene sub-library thereof is constructed as the extracellular recognition domain of CAR; constructing a second genetic element comprising a first cis-regulatory factor, a transcription factor regulated by the first cis-regulatory factor, and a second cis-acting factor regulated by the transcription factor; constructing a third genetic element comprising an inducible protein gene; and inserting the three genetic elements into one or more controlled gene expression cassettes;
C. introduction of the genetic elements into the cells
introducing the one or more controlled gene expression cassettes into the mammalian immune cells through a lentiviral vector system to obtain the CAR cell library;
D. in vitro screening of CAR cells
allowing the CAR cell library to contact an antigen in vitro, and screening and enriching a target CAR cell according to the expression of an inducible protein; and
E. in vivo screening of CAR cells
administering an effective amount of the CAR cell library to an experimental subject, and screening and enriching a target CAR cell according to the expression of an inducible protein.

7. The preparation and screening method according to claim 6, wherein
in steps D and E, after the target CAR cell is screened out and enriched, it further comprises reconstructing a secondary CAR cell library through an antibody engineering process.

8. The preparation and screening method according to claim 6, wherein
in step D, the antigen comprises any one or a combination of at least two selected from the group consisting of a wild-type (WT) cell, a cell transfected with a specific antigen gene, a cell binding to a specific antigen, an antigen dissolved in a medium, an antigen coated on a petri dish, an antigen coated on a microbead, and an antigen coated on a culture scaffold; and
in step E, an in vivo antigen refers to an antigen existing in a living human or animal, and comprises any one or a combination of at least two selected from the group consisting of an in vivo cell, an in vivo lesion cell, an in vivo cell transfected with a specific antigen gene, an in vivo cell infected with a specific pathogen, an in vivo cell binding to a specific antigen, and an in vivo cell transplanted into an animal model.

9. A pharmaceutical composition, comprising an active component and a pharmaceutically acceptable diluent or excipient, wherein the active component comprises any one or a combination of at least two selected from the group consisting of the vector assembly according to claim 1, the CAR cell library according to claim 4, the CAR cell obtained by the screening method according to claim 5, a CAR, and a CAR-derived antibody, and at least one medically or pharmaceutically acceptable carrier.

10. A use of the pharmaceutical composition according to claim 9 in the preparation of a drug, a reagent, or a kit for the diagnosis or treatment of a disease that requires the removal of a disease-associated mediator.
